(19) United States
(12) Patent Application Publication
(10) Pub. No.: US 2011/0059942 A1
(43) Pub. Date: Mar. 10, 2011

(54) Azetidinyl G-Protein Coupled Receptor Agonists

(76) Inventors: Matthew ColinThor Fyfe, Oxford (GB); William Gattrell, Oxford (GB); Colin Peter Sambrook-Smith, Oxford (GB); Simon Andrew Swain, Oxford (GB)

(21) Appl. No.: 12/738,542

(22) PCT Filed: Oct. 20, 2008
(86) PCT No.: PCT/GB2008/050970

§ 371 Date: Nov. 22, 2010

(30) Foreign Application Priority Data
Oct. 18, 2007 (GB) 0720389.6

(51) Int. Cl. A61K 31/397; C07D 205/04; C07D 403/12; C07D 401/14; A61P 3/00; A61P 9/12

(52) U.S. Cl. 514/210.18; 548/952; 514/210.01; 548/518; 514/210.2; 546/268.1; 546/279.1

(57) ABSTRACT

Compounds of formula (I): or pharmaceutically acceptable salts thereof, are agonists of GPR119 and are useful for the treatment of diabetes and as peripheral regulators of satiety, e.g. for the treatment of obesity and metabolic syndrome.

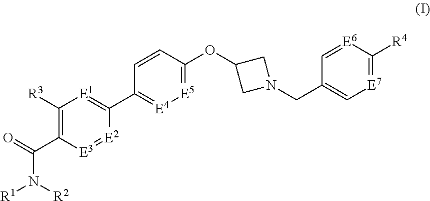

### BACKGROUND OF THE INVENTION

The present invention is directed to G-protein coupled receptor (GPCR) agonists. In particular, the present invention is directed to agonists of GPR119 that are useful for the treatment of obesity, e.g. as regulators of satiety, metabolic syndrome and for the treatment of diabetes.

Obesity is characterized by an excessive adipose tissue mass relative to body size. Clinically, body fat mass is estimated by the body mass index (BMI; weight(kg)/height(m)²), or waist circumference. Individuals are considered obese when the BMI is greater than 30 and there are established medical consequences of being overweight. It has been an accepted medical view for some time that an increased body weight, especially as a result of abdominal body fat, is associated with an increased risk for diabetes, hypertension, heart disease, and numerous other health complications, such as arthritis, stroke, gallbladder disease, muscular and respiratory problems, back pain and even certain cancers.

Pharmacological approaches to the treatment of obesity have been mainly concerned with reducing fat mass by altering the balance between energy intake and expenditure. Many studies have clearly established the link between adiposity and the brain circuitry involved in the regulation of energy homeostasis. Direct and indirect evidence suggest that serotonergic, dopaminergic, adrenergic, cholinergic, endocannabinoid, opioid, and histaminergic pathways in addition to many neuropeptide pathways (e.g. neuropeptide Y and melanocortins) are implicated in the central control of energy intake and expenditure. Hypothalamic centres are also able to sense peripheral hormones involved in the maintenance of body weight and degree of adiposity, such as insulin and leptin, and fat tissue derived peptides.

Drugs aimed at the pathophysiology associated with insulin dependent Type I diabetes and non-insulin dependent Type II diabetes have many potential side effects and do not adequately address the dyslipidaemia and hyperglycaemia in a high proportion of patients. Treatment is often focused at individual patient needs using diet, exercise, hypoglycemic agents and insulin, but there is a continuing need for novel antidiabetic agents, particularly ones that may be better tolerated with fewer adverse effects.

Similarly, metabolic syndrome (syndrome X) places people at high risk of coronary artery disease, and is characterized by a cluster of risk factors including central obesity (excessive fat tissue in the abdominal region), glucose intolerance, high triglycerides and low HDL cholesterol, and high blood pressure. Myocardial ischemia and microvascular disease is an established morbidity associated with untreated or poorly controlled metabolic syndrome.

There is a continuing need for novel antiobesity and antidiabetic agents, particularly ones that are well tolerated with few adverse effects.

GPR119 (previously referred to as GPR116) is a GPCR identified as SNORF25 in WO00/50562 which discloses both the human and rat receptors, U.S. Pat. No. 6,468,756 also discloses the mouse receptor (accession numbers: AAN95194 (human), AAN95195 (rat) and ANN95196 (mouse)).

In humans, GPR119 is expressed in the pancreas, small intestine, colon and adipose tissue. The expression profile of the human GPR119 receptor indicates its potential utility as a target for the treatment of obesity and diabetes.

International patent applications WO2005/061489, WO2006/070208 and WO2006/067532 disclose heterocyclic derivatives as GPR119 receptor agonists. International patent applications WO2006/067531, WO2007/003960, WO2007/003961, WO2007/003962 and WO2007/003964 disclose GPR119 receptor agonists. International patent applications WO2007/116230 and WO2007/116229, published after the priority date of the present application, also disclose GPR119 receptor agonists.

The present invention relates to agonists of GPR119 which are useful for the treatment of diabetes and as peripheral regulators of satiety, e.g. for the treatment of obesity and metabolic syndrome.

### SUMMARY OF THE INVENTION

Compounds of formula (I):

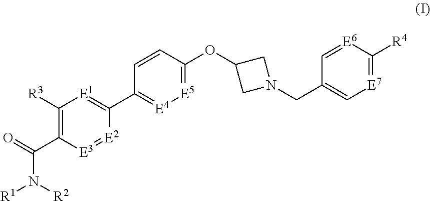

or pharmaceutically acceptable salts thereof, are agonists of GPR119 and are useful for the treatment of diabetes and as peripheral regulators of satiety, e.g. for the treatment of obesity and metabolic syndrome.

### DETAILED DESCRIPTION OF THE INVENTION

The present invention is directed to a compound of formula (I), or a pharmaceutically acceptable salt thereof:

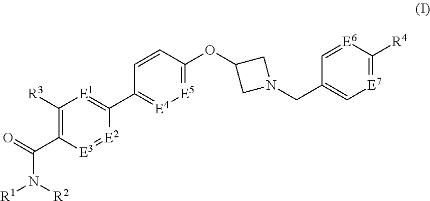

wherein:

E¹, E² and E³ are CH or one of E¹, E² and E³ is N;

E⁴ and E⁵ are CH or one of E⁴ and E⁵ is N;

E⁶ and E⁷ are independently CH or N;

R¹ and R² are independently selected from hydrogen, C₁₋₄ alkyl optionally substituted by one or more hydroxy groups, and a 4- to 6-membered heterocyclic ring containing one heteroatom selected from N and O; or R¹ and R² together with the N to which they are attached may form a 4- to 6-membered heterocyclic ring optionally containing a further heteroatom selected from N and O and optionally substituted by one or more hydroxy or C₁₋₄ alkyl groups;

R³ is hydrogen, halo or methyl; and

R⁴ is C₁₋₄ alkyl or C₁₋₄ alkoxy, either of which may be substituted by one or more fluoro groups.

The molecular weight of the compounds of formula (I) is suitably less than 800, in particular less than 600, especially less than 500.

E¹, E² and E³ are preferably CH.

In one embodiment E⁴ is CH and in another E⁴ is N. Compounds where E⁴ is N may be preferred.

E⁵ are preferably CH.

E⁶ and E⁷ are preferably CH.

R¹ and R² are preferably independently selected from hydrogen and C₁₋₄ alkyl optionally substituted by one or more hydroxy groups; or R¹ and R² together with the N to which they are attached may form a 5- or 6-membered heterocyclic ring optionally containing a further heteroatom selected from N and O and optionally substituted by one or more hydroxy or C₁₋₄ alkyl groups.

R³ is preferably hydrogen or methyl, especially methyl.

R⁴ is preferably C₁₋₄ alkyl or C₁₋₂ alkyl optionally substituted with one or more fluorine atoms, more preferably isopropyl or trifluoromethyl, especially isopropyl.

While the preferred groups for each variable have generally been listed above separately for each variable, preferred compounds of this invention include those in which several or each variable in formula (I) is selected from the preferred, more preferred or particularly listed groups for each variable. Therefore, this invention is intended to include all combinations of preferred, more preferred and particularly listed groups.

Specific compounds of the invention which may be mentioned are those included in the Examples and pharmaceutically acceptable salts thereof.

As used herein, unless stated otherwise, “alkyl” as well as other groups having the prefix “alk” such as, alkoxy, means carbon chains which may be linear or branched or combinations thereof. Examples of alkyl groups include methyl, ethyl, propyl (n-propyl and isopropyl) and butyl (n-butyl, sec-butyl and tert-butyl).

The term “halo” includes fluorine, chlorine, bromine, and iodine atoms.

Unless otherwise indicated the term “heterocyclyl” includes 4- to 6-, e.g. 5- and 6-, membered monocyclic saturated and partially saturated rings containing up to two heteroatoms selected from N and O. Examples of heterocyclic rings include tetrahydrofuran, tetrahydropyran, pyrrolidine, piperidine, [1,3]dioxane, oxazolidine, piperazine, morpholine and the like.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above formula (I) is shown without a definitive stereochemistry at certain positions. The present invention includes all stereoisomers of formula (I) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of formula (I) exists, the present invention includes any possible tautomers and pharmaceutically acceptable salts thereof, and mixtures thereof, except where specifically drawn or stated otherwise.

When the compound of formula (I) and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like

Since the compounds of formula (I) are intended for pharmaceutical use they are preferably provided in substantially pure form, for example at least 60% pure, more suitably at least 75% pure (e.g. 90% or 95%), especially at least 98% pure (% are on a weight for weight basis).

The compounds of formula (I) can be prepared as described below, in which the variable groups are as defined above.

Certain compounds of formula (I) can be made as outlined in Scheme 1. Azetidine 1 is commercially available or can be prepared as outlined in Syn. Comm., 33(24), 4297-4302; 2003. Azetidine 2 can be prepared by treatment of 1 with a hydrogen source such as triethylamine and formic acid, in a solvent such as ethanol in the presence of palladium on carbon. Compounds of type 4 can be prepared by reductive amination of an aldehyde 3 using a suitable reducing agent such as sodium triacetoxyborohydride. Aldehydes of type 3 are commercial, or can be made by readily known techniques. The hydroxy group can be converted into a leaving group such as methanesulfonyl, allowing, in the presence of a base, displacement with a phenol of type 6 to afford the compound of formula (I). Alternatively, a compound of type 4 could be converted into an azetidine of formula (I) directly, via a Mitsonobu reaction with the corresponding phenol 6 by standard techniques.

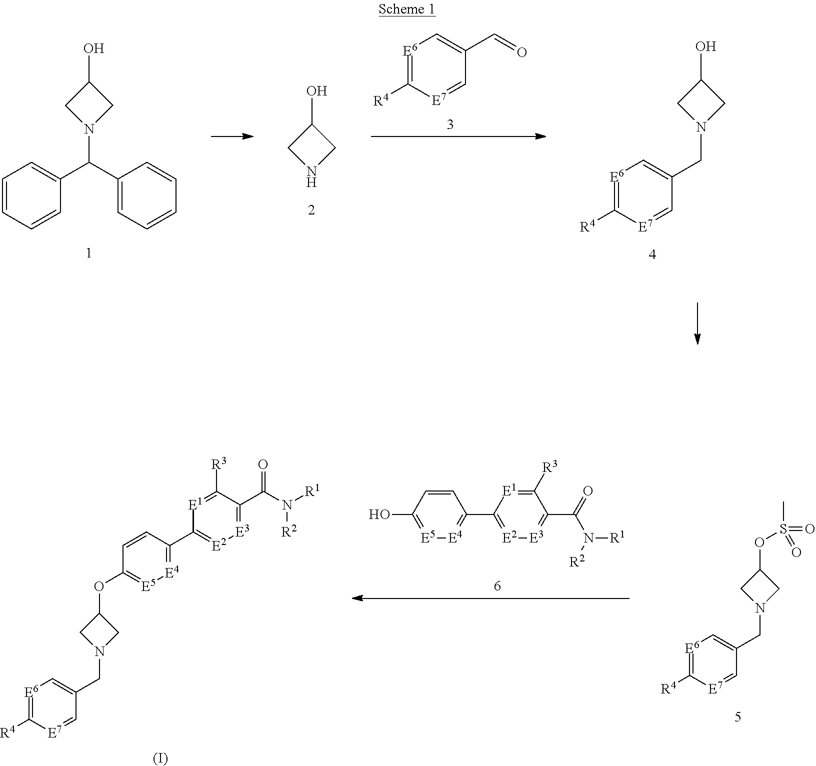

Compounds of type formula (I) can also be prepared as outlined in Scheme 2. Thus, an azetidine of type 7, incorporating a nitrogen protecting group, in this case 2,4-dimethoxybenzyl, can be converted to a compound of formula 9 via an activated azetidine of type 8. Removal of the nitrogen protecting group using standard techniques affords a compound of formula 10. Reductive amination of 10 with an aldehyde of type 3 using a suitable reducing agent affords the compound of formula (I).

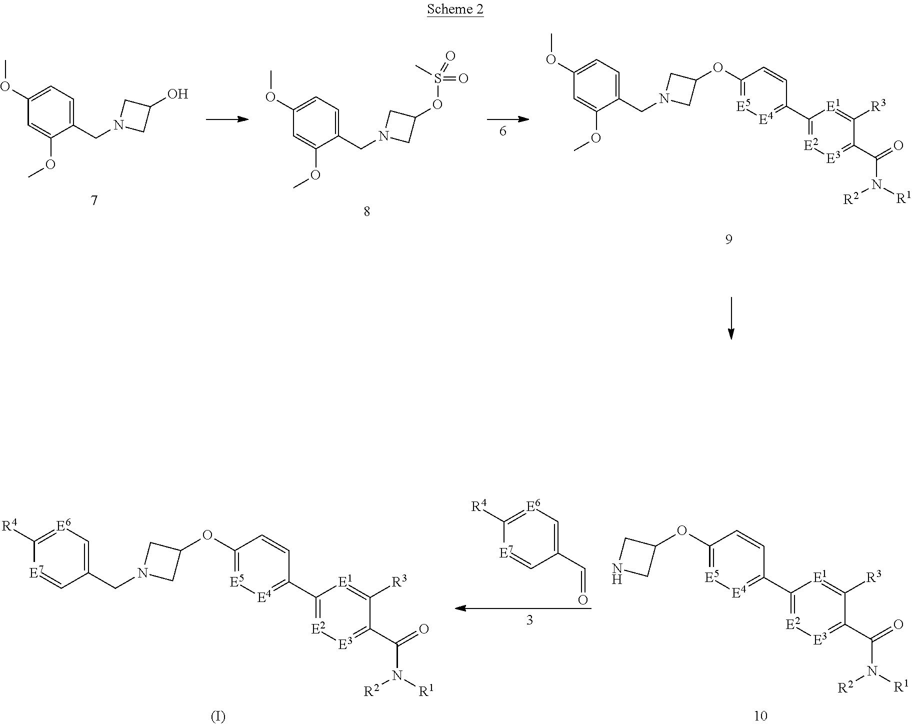

Other compounds of formula (I) can be prepared as outlined in Scheme 3. Thus, an ester of type 11 (where G is a suitable alkyl or aryl group) can be converted to the corresponding carboxylic acid of type 11 (where G is hydrogen) by standard hydrolytic techniques. Formation of compounds (I) by reaction of carboxylic acids of type 11 with amines of type 12 may be facilitated by use of a coupling reagent such as WSC or HATU. Alternatively carboxylic acids of type 11 may be employed in the form of an activated derivative thereof such as an acid halide or acid anhydride. Such activated derivatives may be obtained from the corresponding acid by conventional means. When a compound of type 11 is employed as an acid halide, it may suitably be reacted with an amine in an insert aprotic solvent such as THF in the presence of a base such as TEA.

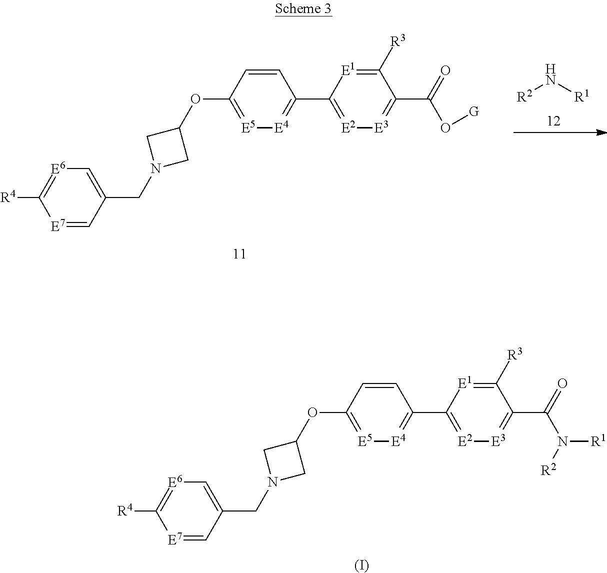

Phenols of type 15 can be prepared using standard Suzuki coupling methods as outlined in Scheme 4. Thus an aryl boronic acid of type 14 (or the corresponding aryl boronic ester) can be coupled with an arylhalide of type 13 (where X is a halogen) using well known Suzuki chemistry. Typically this is carried out in the presence of a suitable base and palladium catalyst in an appropriate solvent or solvent mixture. Alternatively, 13 could incorporate the boronic acid/ester functionality and 14 incorporate the halogen group, with coupling via a Suzuki reaction as described. Ester 15 can be converted to an amide of type 6 as outlined above.

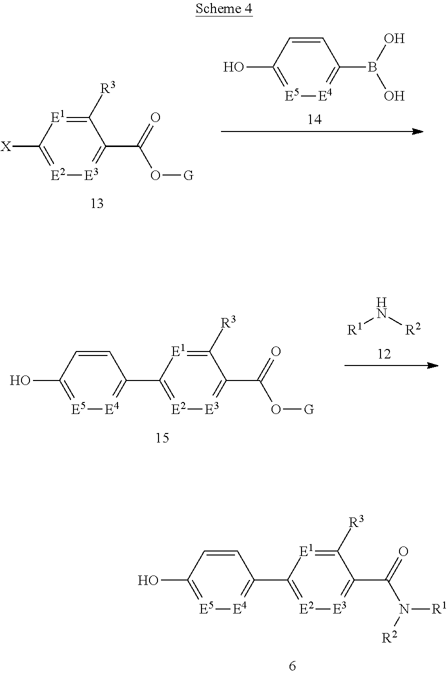

Compounds of type (I) can also be prepared as outlined in Scheme 5. Thus, an aryl halide of type 16 could be reacted with azetidine 4, typically in the presence of a base in an aprotic solvent at elevated temperature. This reaction is most suitable when E⁵, or possibly E⁴, is nitrogen, and the halogen X is fluorine. Aryl halides of type 16 can be prepared using analogous Suzuki chemistry as described in Scheme 4.

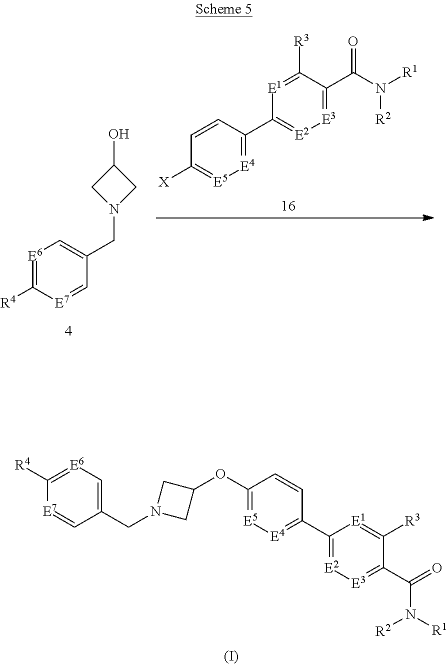

Compounds of formula (I) can be prepared as outlined in Scheme 6. Reaction of phenol 17 with commercially available azetidine 18 would afford a compound of type 19. A reaction of this type would typically employ a base such as sodium tert-butoxide in a polar aprotic solvent. Conversion to compounds of type 20 can be carried out using Suzuki-type chemistry as outlined in Scheme 4. Compounds of formula (I) can be accessed by two approaches from intermediate 20. Either the amide (—NR¹R²) portion can be installed first, i.e. via intermediate 21, with a final step employing aldehyde 3. Alternatively, the group on the azetidine nitrogen can be installed initially, i.e. via intermediate 22, with a final step establishing the amide (—NR′R²) moiety. The chemistry required to install the aldehyde and amine-derived portions is described in Schemes 2 and 3. The nitrogen protecting group, tert-butoxycarbonyl, can be removed as appropriately, by a number of methods, such as treatment with trifluoroacetic acid in an inert solvent.

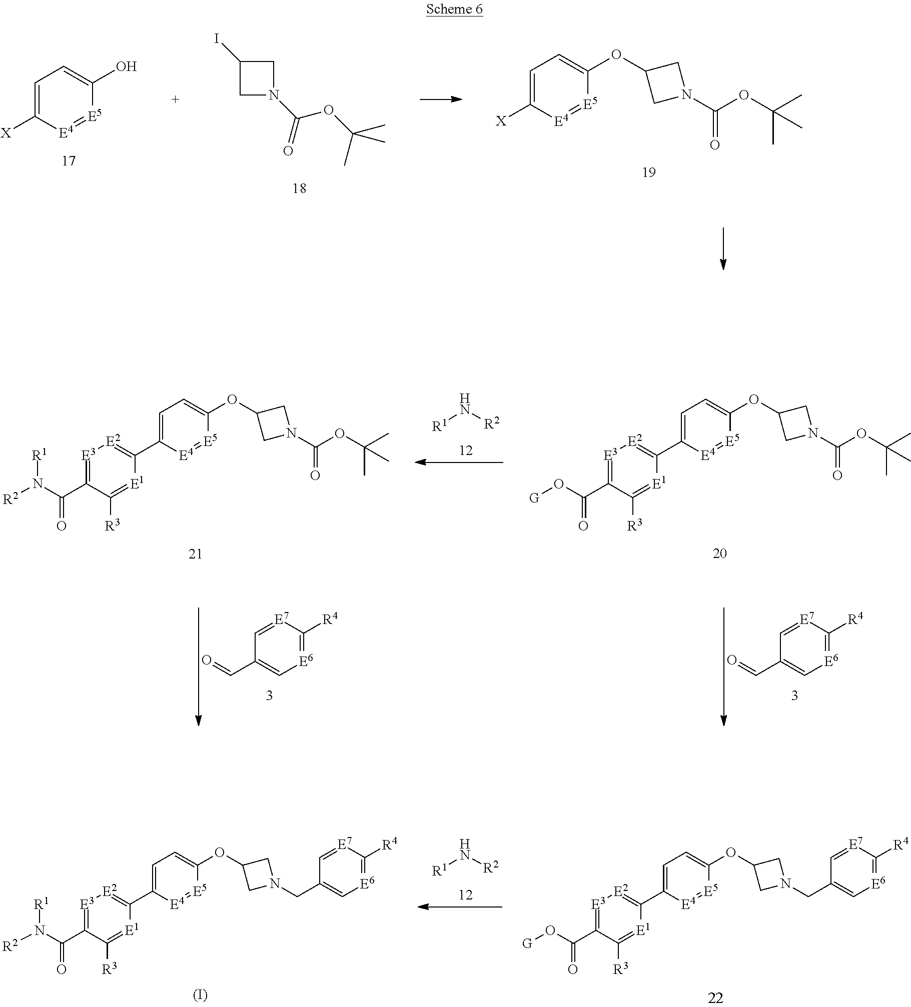

Other compounds of formula (I) may be prepared by methods analogous to those described above or by methods known per se.

Further details for the preparation of the compounds of formula (I) are found in the examples.

The compounds of formula (I) may be prepared singly or as compound libraries comprising at least 2, for example 5 to 1,000, compounds and more preferably 10 to 100 compounds of formula (I). Compound libraries may be prepared by a combinatorial “split and mix” approach or by multiple parallel synthesis using either solution or solid phase chemistry, using procedures known to those skilled in the art.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

Any novel intermediates, such as those defined above, may be of use in the synthesis of compounds of formula (I) and are therefore also included within the scope of the invention.

As indicated above the compounds of formula (I) are useful as GPR119 agonists, e.g. for the treatment and/or prophylaxis of obesity and diabetes. For such use the compounds of formula (I) will generally be administered in the form of a pharmaceutical composition.

The invention also provides a compound of formula (I), or a pharmaceutically acceptable salt thereof, for use as a pharmaceutical.

The invention also provides a pharmaceutical composition comprising a compound of formula (I), in combination with a pharmaceutically acceptable carrier.

Preferably the composition is comprised of a pharmaceutically acceptable carrier and a non-toxic therapeutically effective amount of a compound of formula (I), or a pharmaceutically acceptable salt thereof.

Moreover, the invention also provides a pharmaceutical composition for the treatment of disease by modulating GPR119, resulting in the prophylactic or therapeutic treatment of obesity, e.g. by regulating satiety, or for the treatment of diabetes, comprising a pharmaceutically acceptable carrier and a non-toxic therapeutically effective amount of compound of formula (I), or a pharmaceutically acceptable salt thereof.

The pharmaceutical compositions may optionally comprise other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of formula (I), or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous).

Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The compounds of formula (I), or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a compound of formula (I), or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of formula (I), or pharmaceutically acceptable salts thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of formula (I) may be used in the treatment of diseases or conditions in which GPR119 plays a role.

Thus the invention also provides a method for the treatment of a disease or condition in which GPR119 plays a role comprising a step of administering to a subject in need thereof an effective amount of a compound of formula (I), or a pharmaceutically acceptable salt thereof. Diseases or conditions in which GPR119 plays a role include obesity and diabetes. In the context of the present application the treatment of obesity is intended to encompass the treatment of diseases or conditions such as obesity and other eating disorders associated with excessive food intake e.g. by reduction of appetite and body weight, maintenance of weight reduction and prevention of rebound and diabetes (including Type 1 and Type 2 diabetes, impaired glucose tolerance, insulin resistance and diabetic complications such as neuropathy, nephropathy, retinopathy, cataracts, cardiovascular complications and dyslipidaemia). And the treatment of patients who have an abnormal sensitivity to ingested fats leading to functional dyspepsia. The compounds of the invention may also be used for treating metabolic diseases such as metabolic syndrome (syndrome X), impaired glucose tolerance, hyperlipidemia, hypertriglyceridemia, hypercholesterolemia, low HDL levels and hypertension.

The invention also provides a method for the regulation of satiety comprising a step of administering to a subject in need thereof an effective amount of a compound of formula (I), or a pharmaceutically acceptable salt thereof.

The invention also provides a method for the treatment of obesity comprising a step of administering to a subject in need thereof an effective amount of a compound of formula (I), or a pharmaceutically acceptable salt thereof.

The invention also provides a method for the treatment of diabetes, including type 1 and type 2 diabetes, particularly type 2 diabetes, comprising a step of administering to a patient in need thereof an effective amount of a compound of formula (I), or a pharmaceutically acceptable salt thereof.

The invention also provides a method for the treatment of metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound of formula (I), or a pharmaceutically acceptable salt thereof.

The invention also provides a compound of formula (I), or a pharmaceutically acceptable salt thereof, for use in the treatment of a condition as defined above.

The invention also provides the use of a compound of formula (I), or a pharmaceutically acceptable salt thereof, in the manufacture of a medicament for the treatment of a condition as defined above.

In the methods of the invention the term “treatment” includes both therapeutic and prophylactic treatment.

The compounds of formula (I) may exhibit advantageous properties compared to known GPR119 agonists, for example, the compounds may exhibit improved in vivo activity, improved solubility thus improving absorption properties and bioavailability, or other advantageous properties for compounds to be used as pharmaceuticals.

The compounds of formula (I), or pharmaceutically acceptable salts thereof, may be administered alone or in combination with one or more other therapeutically active compounds. The other therapeutically active compounds may be for the treatment of the same disease or condition as the compounds of formula (I) or a different disease or condition. The therapeutically active compounds may be administered simultaneously, sequentially or separately.

The compounds of formula (I) may be administered with other active compounds for the treatment of obesity and/or diabetes, for example insulin and insulin analogs, gastric lipase inhibitors, pancreatic lipase inhibitors, sulfonyl ureas and analogs, biguanides, α2 agonists, glitazones, PPAR-γ agonists, mixed PPAR-α/γ agonists, DPIV inhibitors, RXR agonists, fatty acid oxidation inhibitors, α-glucosidase inhibitors, β-agonists, phosphodiesterase inhibitors, lipid lowering agents, glycogen phosphorylase inhibitors, antiobesity agents e.g. pancreatic lipase inhibitors, MCH-1 antagonists and CB-1 antagonists (or inverse agonists), amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, serotonergic/dopaminergic antiobesity drugs, reuptake inhibitors e.g. sibutramine, CRF antagonists, CRF binding proteins, thyromimetic compounds, aldose reductase inhibitors, glucocorticoid receptor antagonists, NHE-1 inhibitors or sorbitol dehydrogenase inhibitors.

Combination therapy comprising the administration of a compound of formula (I), or a pharmaceutically acceptable salt thereof, and at least one other antiobesity agent represents a further aspect of the invention.

The present invention also provides a method for the treatment of obesity in a mammal, such as a human, which method comprises administering an effective amount of a compound of formula (I), or a pharmaceutically acceptable salt thereof, and another antiobesity agent, to a mammal in need thereof.

The invention also provides the use of a compound of formula (I), or a pharmaceutically acceptable salt thereof, and another antiobesity agent for the treatment of obesity.

The invention also provides the use of a compound of formula (I), or a pharmaceutically acceptable salt thereof, in the manufacture of a medicament for use in combination with another antiobesity agent, for the treatment of obesity.

The compound of formula (I), or a pharmaceutically acceptable salt thereof, and the other antiobesity agent(s) may be co-administered or administered sequentially or separately.

Co-administration includes administration of a formulation which includes both the compound of formula (I), or a pharmaceutically acceptable salt thereof, and the other antiobesity agent(s), or the simultaneous or separate administration of different formulations of each agent. Where the pharmacological profiles of the compound of formula (I), or a pharmaceutically acceptable salt thereof, and the other antiobesity agent(s) allow it, coadministration of the two agents may be preferred.

The invention also provides the use of a compound of formula (I), or a pharmaceutically acceptable salt thereof, and another antiobesity agent in the manufacture of a medicament for the treatment of obesity.

The invention also provides a pharmaceutical composition comprising a compound of formula (I), or a pharmaceutically acceptable salt thereof, and another antiobesity agent, and a pharmaceutically acceptable carrier. The invention also encompasses the use of such compositions in the methods described above.

GPR119 agonists are of particular use in combination with centrally acting antiobesity agents.

The other antiobesity agent for use in the combination therapies according to this aspect of the invention is preferably a CB-1 modulator, e.g. a CB-1 antagonist or inverse agonist. Examples of CB-1 modulators include SR141716 (rimonabant) and SLV-319 ((4S)-(−)-3-(4-chlorophenyl)-N-methyl-N-[(4-chlorophenyl)sulfonyl]-4-phenyl-4,5-dihydro-1H-pyrazole-1-carboxamide); as well as those compounds disclosed in EP576357, EP656354, WO 03/018060, WO 03/020217, WO 03/020314, WO 03/026647, WO 03/026648, WO 03/027076, WO 03/040105, WO 03/051850, WO 03/051851, WO 03/053431, WO 03/063781, WO 03/075660, WO 03/077847, WO 03/078413, WO 03/082190, WO 03/082191, WO 03/082833, WO 03/084930, WO 03/084943, WO 03/086288, WO 03/087037, WO 03/088968, WO 04/012671, WO 04/013120, WO 04/026301, WO 04/029204, WO 04/034968, WO 04/035566, WO 04/037823 WO 04/052864, WO 04/058145, WO 04/058255, WO 04/060870, WO 04/060888, WO 04/069837, WO 04/069837, WO 04/072076, WO 04/072077, WO 04/078261 and WO 04/108728, and the references disclosed therein.

Other diseases or conditions in which GPR119 has been suggested to play a role include those described in WO 00/50562 and U.S. Pat. No. 6,468,756, for example cardiovascular disorders, hypertension, respiratory disorders, gestational abnormalities, gastrointestinal disorders, immune disorders, musculoskeletal disorders, depression, phobias, anxiety, mood disorders and Alzheimer's disease.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### EXAMPLES

LCMS data were obtained as follows. Unless otherwise stated, LCMS Method A was employed.

### LCMS Method A:

Waters Atlantis C18, 3μ (3.0×20 mm, flow rate 0.85 mL/min) eluting with a H₂O-MeCN gradient containing 0.1% v/v HCO₂H over 6.5 min with UV detection at 220 nm. Gradient information: 0.0-0.3 min 100% H₂O; 0.3-4.25 min: Ramp to 10% H₂O-90% CH₃CN; 4.25 min-4.4 min: Ramp to 100% CH₃CN; 4.4-4.9 min: Hold at 100% MeCN; 4.9-5.0 min: Return to 100% H₂O; 5.00-6.50 min: Hold at 100% H₂O. The mass spectra were obtained using an electrospray ionisation source in either the positive (ESI⁺) ion or negative ion (ESI⁻) mode.

### LCMS Method B:

Agilent Prep-C18 Scalar column, 5 μm (4.6×50 mm, flow rate 2.5 mL/min) eluting with a H₂O-MeCN gradient containing 0.1% v/v formic acid over 7 minutes with UV detection at 254 nm. Gradient information: 0.0-0.5 min: 95% H₂O-5% MeCN; 0.5-5.0 min; Ramp from 95% H₂O-5% MeCN to 5% H₂O-95% MeCN; 5.0-5.5 min: Hold at 5% H₂O-95% MeCN; 5.5-5.6 min: Hold at 5% H₂O-95% MeCN, flow rate increased to 3.5 mL/min; 5.6-6.6 min: Hold at 5% H₂O-95% MeCN, flow rate 3.5 mL/min; 6.6-6.75 min: Return to 95% H₂O-5% MeCN, flow rate 3.5 mL/min; 6.75-6.9 min: Hold at 95% H₂O-5% MeCN, flow rate 3.5 mL/min; 6.9-7.0 min: Hold at 95% H₂O-5% MeCN, flow rate reduced to 2.5 mL/min. Mass spectra were obtained using an Agilent multimode source in either the positive (APCI+ESI⁺) or negative (APCI+ESI⁻) mode.

Abbreviations and acronyms: Ac: acetyl; DCM: dichloromethane; DCE: dichloroethane; DIPEA: diisopropylethylamine; DMAP: 4-dimethylaminopyridine; DME: 1,2-dimethoxyethane; DMSO: dimethylsulfoxide; EDCI: 1-(3-dimethylaminopropyl)-3-ethylcarbodiimide hydrochloride; Et: ethyl; HATU: O-(7-azabenzotriazol-1-yl)-N,N,N′,N′-tetramethyluronium hexafluorophosphate; h: hour(s); min: minute/s; HOBt: 1-hydroxybenzotriazole; HPLC: high performance liquid chromatography; IH: isohexane; IPA: iso-propyl alcohol; LCMS: liquid chromatography-mass spectrometry; Me: methyl; RT: retention time; SCX: strong cation exchange chromatography; WSC: water soluble carbodiimide hydrochloride.

The syntheses of the following compounds have been described elsewhere: (4-bromo-2-methylphenyl)pyrrolidin-1-yl-methanone: WO2006/021759; 4-(1,1-difluoroethyl)-benzaldehyde: Tetrahedron, 1975, 31(5), 391-401. All other compounds were available from commercial sources.

### Preparation 1: Azetidin-3-ol

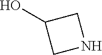

To a solution of 1-benzhydrylazetidin-3-ol (30.5 g, 130 mmol) in ethanol (500 mL) was added a pre-mixed solution of triethylamine (55 mL, 390 mmol) and formic acid (15 mL, 390 mol) in ethanol (100 mL). Palladium on carbon (2.40 g) was added and the mixture heated to reflux for 3 h. The mixture was cooled to ambient temperature and filtered through celite to afford the title compound as a solution in ethanol.

### Preparation 2: 1-(4-Isopropylbenzyl)azetidin-3-ol

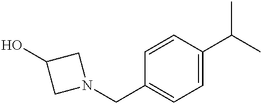

To a solution of azetidin-3-ol (Preparation 1, 6.84 mmol) and 4-isopropylbenzaldehyde (8.21 mmol) in ethanol (45 mL) was added acetic acid (0.5 mL). After stirring for 1 h, sodium triacetoxyborohydride (8.21 mmol) was added, stirring continued for 3 days. Aqueous hydrochloric acid (1M, 30 mL) was added and the mixture concentrated to remove ethanol. The mixture was extracted with diethyl ether (×2), and the remaining aqueous mixture basified by addition of 2M sodium hydroxide. The solution was then extracted with DCM (×3). The combined DCM extracts were dried (MgSO₄) and concentrated to afford the title compound; RT=2.00 min; m/z (ES⁺)=206.07 [M+H]⁺.

### Preparation 3: Methanesulfonic acid 1-(4-isopropylbenzyl)azetidin-3-yl ester

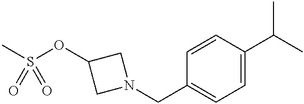

To a solution of 1-(4-isopropylbenzyl)-azetidin-3-ol (Preparation 2, 1.34 mmol) and triethylamine (2.95 mmol) in DCM (5 mL) at 0° C. was added methane sulfonyl chloride (1.61 mmol). The stirred mixture was then allowed to reach ambient temperature over a period of 2 h. The mixture was then diluted with further DCM and washed with saturated sodium carbonate solution, dried (MgSO₄) and concentrated to afford the title compound; RT=2.50 min; m/z (ES⁺)=284.12 [M+H]⁺.

### Preparation 4: 4′-Hydroxybiphenyl-4-carboxylic acid ethyl ester

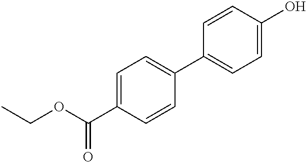

A suspension of 4′-hydroxybiphenyl-4-carboxylic acid (20.7 mmol) and concentrated sulphuric acid (1 mL) in ethanol was heated under reflux for 6 h. The mixture was cooled, concentrated to remove the majority of ethanol and the resulting solution neutralised by addition of 2M aqueous sodium hydroxide. The solution was basified by addition of saturated sodium hydrogen carbonate solution and diluted with water. The resulting solid was filtered and dried to obtain the title compound; RT=3.54 min; m/z (ES⁺)=243.10 [M+H]⁺.

The following intermediate was prepared in a similar manner:

### Preparation 5: 4′-Hydroxy-3-methylbiphenyl-4-carboxylic acid ethyl ester; RT 3.65 min; m/z (ES⁺)=257.13 [M+H]⁺.

### Preparation 6: 4′-Hydroxy-3-methylbiphenyl-4-carboxylic acid

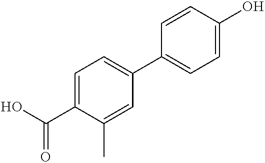

A mixture of 4-bromo-2-methylbenzoic acid (6.57 mmol), 4-(4,4,5,5-tetramethyl-[1,3,2]dioxaborolan-2-yl)phenol (7.20 mmol), caesium carbonate (19.5 mmol) and [1,1′-bis(diphenylphosphino)ferrocene]dichloropalladium(II) (650 μmol) were combined under argon in dioxane/water (4:1, 20 mL). The stirred mixture was heated under reflux for 17 h. The mixture was cooled, concentrated and acidified to pH 6 by addition of 1M hydrochloric acid. The mixture was then extracted with ethyl acetate (×3), and the combined organic extracts dried (MgSO₄) and concentrated. Flash chromatography afforded the title compound; RT=3.07 min.

### Preparation 7: 4′-[1-(4-Isopropylbenzyl)azetidin-3-yloxy]biphenyl-4-carboxylic acid ethyl ester

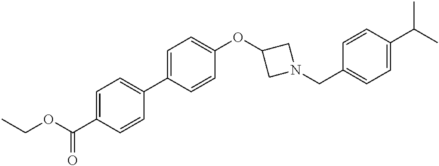

To a solution of 4′-hydroxybiphenyl-4-carboxylic acid ethyl ester (Preparation 4, 10.1 mmol) in anhydrous DMSO (15 mL) was added potassium tert-butoxide (10.5 mmol). After 20 min, methanesulfonic acid 1-[1-(4-iso-propylphenyl)ethyl]azetidin-3-yl ester (Preparation 3, 0.44 mmol) was added and the mixture heated to 60° C. for 1 h. The mixture was cooled, diluted with DCM/diethyl ether and washed with brine. The organic extract was dried (MgSO₄) and concentrated. Flash chromatography afforded the title compound; RT=3.30 min; m/z (ES⁺)=430.220 [M+H]⁺.

### Preparation 8: 4′-[1-(4-Isopropylbenzyl)azetidin-3-yloxy]biphenyl-4-carboxylic acid

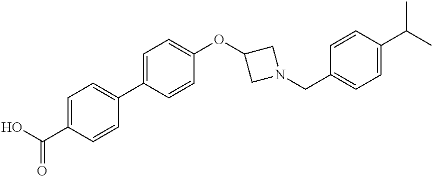

To a solution of 4′41-(4-isopropylbenzyl)azetidin-3-yloxy]biphenyl-4-carboxylic acid ethyl ester (Preparation 7, 1.16 mmol) in methanol (15 mL) was added sodium hydroxide (2M, 5 mL). After stirring at ambient temperature for 6 h the mixture was heated to 50° C. for 3 days. The mixture was cooled, extracted with ethyl acetate and neutralised by addition of 2M hydrochloric acid. The resulting solid was washed with ethyl acetate and concentrated to afford the title compound, used without further purification; RT=2.85 min; m/z (ES⁺)=402.19 [M+H]⁺.

### Preparation 9: 1-Chloro-3-(4-trifluoromethylbenzylamino)propan-2-ol

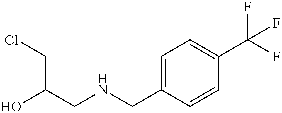

Epichlorohydrin (26.0 g, 281 mmol) was added to a solution of 4-trifluoromethyl-benzylamine (50.0 g, 286 mmol) in IPA:IH (9:1, 300 mL) and the resulting solution was stirred at ambient temperature for 16 h. The solvent was removed in vacuo and the remainder was triturated with IH (150 mL). The resulting white solid was collected by filtration and washed with IH (2×50 mL) to afford the title compound: δ_{H} (CDCl₃) 2.43 (br s, 2H), 2.71-2.80 (m, 1H), 2.82-2.90 (m, 1H), 3.60 (d, 2H), 3.88-3.97 (m, 3H), 7.45 (d, 2H), 7.61 (d, 2H).

### Preparation 10:1-(4-Trifluoromethylbenzyl)azetidin-3-ol

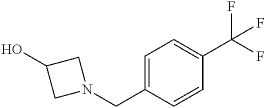

Tetrabutylammonium iodide (3.00 g, 8.11 mmol) was added to a solution of 1-chloro-3-(4-trifluoromethylbenzylamino)propan-2-ol (Preparation 9, 67.7 g, 253 mmol) in triethylamine (340 mL) and the resulting reaction mixture was stirred at 75° C. for 62 h. The reaction mixture was cooled to ambient temperature, filtered and the filtrate concentrated in vacuo, azeotroping with toluene (2×150 mL). The remainder was triturated with IH and the resulting solid was suspended in IH:DIPEA (19:1, 100 mL) and stirred for 2 h. The solid was collected by filtration and washed with IH:DIPEA (19:1, 52 mL) to afford the title compound: δ_{H} (CDCl₃) 2.47 (br s, 1H), 2.92-3.00 (m, 2H), 3.61-3.70 (m, 4H), 4.47 (quin, 1H), 7.39 (d, 2H), 7.57 (d, 2H).

### Preparation 11: 3-(4-Bromophenoxy)-1-(4-trifluoromethylbenzyl)azetidine

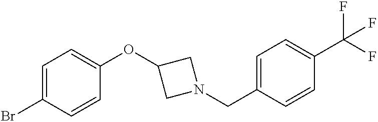

Methanesulfonylchloride (560 μL, 7.14 mmol) was added to a solution of 1-(4-trifluoromethylbenzyl)azetidin-3-ol (Preparation 10, 1.50 g, 6.50 mmol) and triethylamine (1.00 mL, 7.14 mmol) in THF (2 mL) at 0° C. The resulting reaction mixture was stirred at this temperature for 20 min before adding to a solution of 4-bromophenol (1.13 g, 6.50 mmol) and NaH (1.04 g, 26.0 mmol) in DMF (5 mL). The resulting reaction mixture was stirred at ambient temperature for 16 h and then at 45° C. for 16 h, before quenching with H₂O. The reaction mixture was extracted with DCM and the organic extract washed with 1M NaOH and brine, dried (MgSO₄), filtered and concentrated in vacuo. Purification by column chromotography (EtOAc:IH, 3:7) afforded the title compound: RT=2.88 min; m/z (ES⁺)=388.00 [M+H]⁺.

### Preparation 12: 5-(4,4,5,5-Tetramethyl-[1,3,2]dioxaborolan-2-yl)pyridine-2-carboxylic acid ethylamide

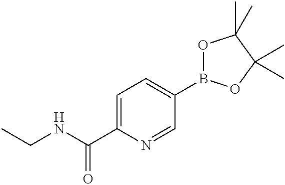

Dioxane (40 mL) was added to 5-bromopyridine-2-carboxylic acid ethylamide (2.50 g, 10.9 mmol), 4,4,5,5,4′,4′,5′,5′-octamethyl-[2,2′]bi[[1,3,2]dioxaborolanyl] (2.77 g, 10.9 mmol), potassium acetate (2.46 g, 25.1 mmol), [1,1′-bis(diphenylphosphino)ferrocene]-dichloropalladium (178 mg, 220 μmol) and 1,1′-bis(diphenylphosphino)ferrocene (121 mg, 220 μmol) and the resulting reaction mixture was degassed with argon and heated at 110° C. for 16 h. The reaction mixture was cooled, partitioned between Et₂O (150 mL) and 1M NaOH (100 mL) and filtered through celite. The organic layer was separated and extracted with 1M NaOH (2×50 mL). The aqueous layers were combined, washed with Et₂O (100 mL), acidified to pH 6 with 12M HCl and extracted with EtOAc (4×100 mL). The combined EtOAc extracts were dried (MgSO₄), filtered and concentrated in vacuo. Purification by column chromatography (EtOAc—IH, 7:3) afforded the title compound: RT=1.97 min; m/z (ES⁺)=195.06 [M+H]⁺.

### Preparation 13: 3-[4-(4,4,5,5-Tetramethyl-[1,3,2]dioxaborolan-2-yl)phenoxy]-1-(4-trifluoromethylbenzyl)azetidine

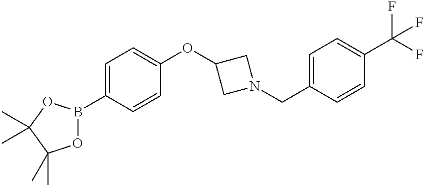

The title compound was synthesized from 3-(4-bromophenoxy)-1-(4-trifluoromethyl-benzyl)azetidine (Preparation 11, 690 mg, 1.78 mmol) employing a procedure similar to that outlined in Preparation 12: RT=3.09 min; m/z (ES⁺)=434.33 [M+H]⁺.

### Preparation 14: 2-Methyl-6-trifluoromethanesulfonyloxynicotinic acid ethyl ester

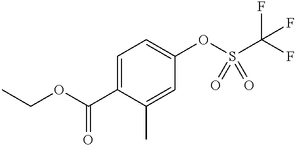

N-Phenyl-bis(trifluoromethylsulfonimide) (3.55 g, 9.90 mmol) was added to a suspension of 6-hydroxy-2-methylnicotinic acid ethyl ester (1.50 g, 8.30 mmol), triethylamine (2.31 mL, 16.6 mmol) and DMAP (10.0 mg, 81.9 μmol) in DCM (30 mL) and the resulting reaction mixture was stirred at ambient temperature for 16 h. The reaction mixture was diluted with DCM (30 mL), washed with H₂O (10 mL) and brine (10 mL), dried (MgSO₄), filtered and concentrated in vacuo. Purification by column chromatography (EtOAc—IH, 1:19 to 1:9) afforded the title compound: RT=3.95 min; m/z (ES⁺)=314.11 [M+H]⁺.

### Preparation 15: 2-Methyl-6-{4-[1-(4-trifluoromethylbenzyl)azetidin-3-yloxy]phenyl}nicotinic acid ethyl ester

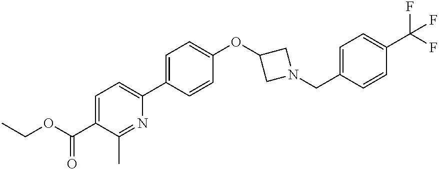

The title compound was synthesized from 3-[4-(4,4,5,5-tetramethyl-[1,3,2]dioxaborolan-2-yl)phenoxy]-1-(4-trifluoromethylbenzyl)azetidine (Preparation 13, 512 mg, 1.18 mmol) and 2-methyl-6-trifluoromethanesulfonyloxynicotinic acid ethyl ester (Preparation 14, 370 mg, 1.18 mmol) employing a procedure similar to that outlined in Example 9: RT=3.15 min; m/z (ES⁺)=471.39 [M+H]⁺.

### Preparation 16: 2-Methyl-6-{4-[1-(4-trifluoromethylbenzyl)azetidin-3-yloxy]phenyl}nicotinic sodium carboxylate

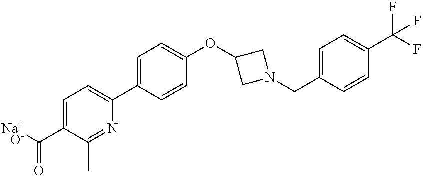

NaOH (23.2 mg, 580 μmol) in H₂O (1.16 mL) was added to a suspension of 2-methyl-6-{4-[1-(4-trifluoromethylbenzyl)azetidin-3-yloxy]phenyl}nicotinic acid ethyl ester (Preparation 15, 271 mg, 580 μmol) in MeOH (12 mL) and the resulting reaction mixture was stirred at 70° C. for 16 h. The solvent was removed in vacuo to afford the title compound: RT=2.52 min; m/z (ES⁺)=443.34 [M+H]⁺.

### Preparation 17: Methanesulfonic acid 1-(4-trifluoromethylbenzyl)azetidin-3-yl ester

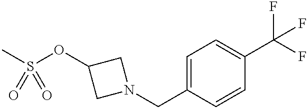

Methanesulfonylchloride (241 μL, 3.11 mmol) was added to a solution of 1-(4-trifluoromethylbenzyl)azetidin-3-ol (Preparation 10, 600 mg, 2.59 mmol) and triethylamine (794 μL, 5.70 mmol) in DCM (10 mL) at 0° C. The resulting reaction mixture was stirred at ambient temperature for 1.5 h. The reaction mixture was diluted with DCM (100 mL), washed with saturated aqueous Na₂CO₃ solution (2×50 mL), dried (MgSO₄), filtered and concentrated in vacuo to afford the title compound: RT=1.99 min; m/z (ES⁺)=310.05 [M+H]⁺.

### Preparation 18: 2-Bromo-5-[1-(4-trifluoromethylbenzyl)azetidin-3-yloxy]pyridine

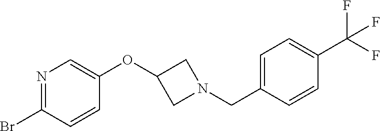

Potassium tertiary butoxide (3.08 g, 27.4 mmol) was added to a solution of 6-bromo-pyridin-3-ol (4.78 g, 27.4 mmol) in DMSO (100 mL) and the resulting solution was stirred at ambient temperature for 20 min prior to the addition of methanesulfonic acid 1-(4-trifluoromethylbenzyl)azetidin-3-yl ester (Preparation 17, 10.6 g, 34.3 mmol) in DMSO (20 mL). The resulting reaction mixture was heated at 100° C. for 3 h before pouring into H₂O (300 mL) and extracting with DCM (2×200 mL). The combined organic extracts were washed with 2M NaOH (2×100 mL), dried (MgSO₄), filtered and concentrated in vacuo. Purification by column chromotography (EtOAc—IH, 7:13) afforded the title compound: RT=2.49 min; m/z (ES⁺)=387.00, 389.00 [M+H]⁺.

### Preparation 19: [2-Methyl-4-(4,4,5,5-tetramethyl-[1,3,2]dioxaborolan-2-yl)phenyl]pyrrolidin-1-ylmethanone

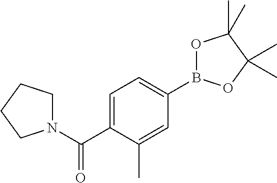

The title compound was synthesized from (4-bromo-2-methylphenyl)pyrrolidin-1-yl-methanone (710 mg, 2.65 mmol) employing a procedure similar to that outlined in Preparation 12: RT=3.55 min; m/z (ES⁺)=316.30 [M+H]⁺.

### Preparation 20: 4-Bromo-N-ethyl-2-methylbenzamide

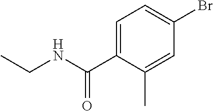

A solution of 4-bromo-2-methylbenzoic acid (5.00 g, 23.3 mmol), EDCI (4.90 g, 25.9 mmol), HOBt (3.46 g, 25.9 mmol), triethylamine (9.40 g, 93.0 mmol) and ethylamine hydrochloride (2.84 g, 34.9 mmol) were stirred at 50° C. for 16 h. The solvent was removed in vacuo and the remainder was dissolved in EtOAc and washed with saturated aqueous NaHCO₃ solution and brine, dried (MgSO₄), filtered and concentrated in vacuo to afford the title compound: δ_{H} (CDCl₃) 1.25 (t, 3H), 2.42 (s, 3H), 3.42-3.52 (m, 2H), 5.73 (br s, 1H), 7.21 (d, 1H), 7.30-7.41 (m, 2H).

### Preparation 21: N-Ethyl-2-methyl-4-(4,4,5,5-tetramethyl-[1,3,2]dioxaborolan-2-yl)benzamide

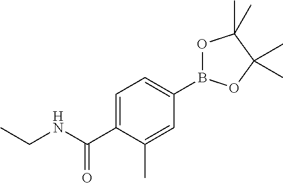

The title compound was synthesized from 4-bromo-N-ethyl-2-methylbenzamide (Preparation 20, 500 mg, 2.07 mmol) employing a procedure similar to that outlined in Preparation 12: RT=3.27 min; m/z (ES⁺)=290.26 [M+H]⁺.

### Preparation 22: 3-(6-Bromopyridin-3-yloxy)azetidine-1-carboxylic acid tert-butyl ester

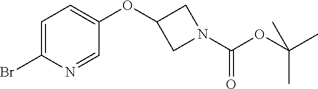

Sodium tertiary butoxide (276 mg, 2.87 mmol) and potassium benzoate (460 mg, 2.87 mmol) were added to a solution of 6-bromo-pyridin-3-ol (500 mg, 2.87 mmol) and 3-iodo-azetidine-1-carboxylic acid tert-butyl ester in DMSO (10 mL). The resulting reaction mixture was stirred at 50° C. for 2 h. Further 3-iodo-azetidine-1-carboxylic acid tert-butyl ester (407 mg, 1.44 mmol) was added and heating at 50° C. was continued for 2 h. The reaction mixture was cooled to ambient temperature, diluted with EtOAc (100 mL) and washed with saturated aqueous Na₂CO₃ solution (2×30 mL). The combined aqueous washings were extracted with EtOAc (2×70 mL) and the combined organic extracts were washed with brine, dried (MgSO₄), filtered and concentrated in vacuo. Purification by column chromatography (EtOAc—IH, 1:4) afforded the title compound: RT=3.52 min; m/z (ES⁺)=329.18 [M+H]⁺.

### Preparation 23: 3-[6-(4-Ethoxycarbonyl-3-fluorophenyl)pyridin-3-yloxy]azetidine-1-carboxylic acid tert-butyl ester

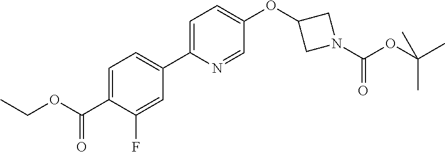

The title compound was synthesized from 3-(6-bromopyridin-3-yloxy)azetidine-1-carboxylic acid tert-butyl ester (Preparation 22, 2.43 g, 7.38 mmol) and 4-boronic acid-2-fluoro-benzoic acid ethyl ester (1.72 g, 8.12 mmol) employing a procedure similar to that outlined in Example 9: RT=4.15 min; m/z (ES⁺)=417.33 [M+H]⁺.

### Preparation 24: 3-{6-[3-Fluoro-4-(2-hydroxyethylcarbamoyl)phenyl]pyridin-3-yloxy}-azetidine-1-carboxylic acid tert-butyl ester

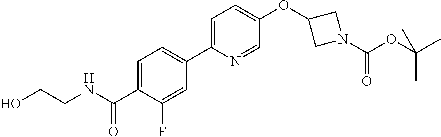

3-[6-(4-Ethoxycarbonyl-3-fluorophenyl)pyridin-3-yloxy]azetidine-1-carboxylic acid tert-butyl ester (Preparation 23, 675 mg, 1.60 mmol) was dissolved in MeOH-Ethanolamine (1:1, 10 mL) and the resulting reaction mixture was heated under reflux conditions for 2 h. The reaction mixture was cooled to ambient temperature, diluted with DCM (100 mL), washed with H₂O (50 mL), dried (MgSO₄), filtered and concentrated in vacuo to afford the title compound: RT=3.20 min; m/z (ES⁺)=432.31 [M+H]⁺.

### Preparation 25: 4-[5-(Azetidin-3-yloxy)pyridin-2-yl]-2-fluoro-N-(2-hydroxyethyl)benzamide hydrochloride

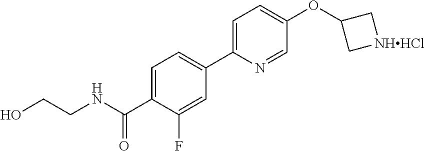

4M HCl in dioxane (5 mL) was added to a solution of 3-{6-[3-fluoro-4-(2-hydroxy-ethylcarbamoyl)phenyl]pyridin-3-yloxy}azetidine-1-carboxylic acid tert-butyl ester (Preparation 24, 612 mg, 1.42 mmol) in MeOH and the resulting reaction mixture was stirred at ambient temperature for 16 h. The solvent was removed in vacuo to afford the title compound: RT=1.92 min; m/z (ES⁺)=332.18 [M+H]⁺.

### Preparation 26: 5-Bromo-2-[1-(4-trifluoromethylbenzyl)azetidin-3-yloxy]pyridine

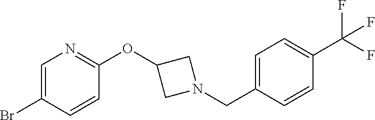

NaH (374 mg, 15.6 mmol) was added to a solution of 1-(4-trifluoromethylbenzyl)-azetidin-3-ol (Preparation 10, 5.01 g, 15.6 mmol) in THF (30 mL) and the resulting reaction mixture was stirred at 0° C. for 1.5 h prior to the addition of 5-bromo-2-chloro-pyridine (3.00 g, 15.6 mmol). The resulting reaction mixture was heated at 65° C. for 4 h before cooling to ambient temperature, quenching with H₂O and removing the solvent in vacuo. The remainder was dissolved in EtOAc, washed with saturated aqueous NaHCO₃ solution, dried (MgSO₄), filtered and concentrated in vacuo. Purification by column chromotography (EtOAc—IH, 1:1) afforded the title compound: RT=2.62 min; m/z (ES⁺)=386.99, 388.99 [M+H]⁺.

### Preparation 27: 2-Fluoro-4-{5-[1-(4-trifluoromethylbenzyl)azetidin-3-yloxy]pyridin-2-yl}-benzoic acid ethyl ester

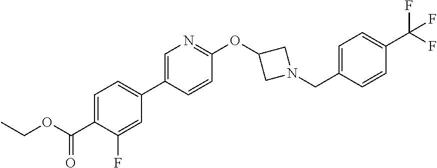

The title compound was synthesized from 2-bromo-5-[1-(4-trifluoromethylbenzyl)-azetidin-3-yloxy]pyridine (Preparation 18, 612 mg, 1.58 mmol) and 4-ethoxycarbonyl-3-fluorophenylboronic acid (369 mg, 1.74 mmol) employing a procedure similar to that outlined in Example 9: RT=3.03 min; m/z (ES⁺)=475.38 [M+H]⁺.

### Preparation 28: 4-(6-Fluoropyridin-3-yl)benzoic acid

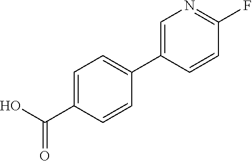

Palladium-tetrakis(triphenylphosphine) (348 mg, 300 μmol) was added to a solution of 4-boronic acid-benzoic acid (1.00 g, 6.03 mmol), 5-bromo-2-fluoropyridine (1.06 g, 6.03 mmol) and 0.4 M Na₂CO₃ solution (25 mL) in MeCN (25 mL) and the resulting reaction mixture was heated at 90° C. for 3 h. The reaction mixture was filtered and the filtrate was concentrated in vacuo to remove the MeCN. The aqueous remainder was washed with DCM (50 mL), acidified with 1M HCl and the resulting precipitate collected by filtration. Purification by recrystallisation afforded the title compound: RT=2.13 min; m/z (ES⁺)=217.99 [M+H]⁺

### Preparation 29: 5-(4-Hydroxyphenyl)-3-methylpyridine-2-carboxylic acid

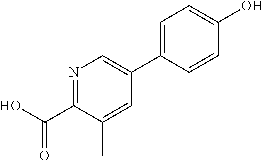

5-Bromo-3-methylpyridine-2-carboxylic acid (5.00 g, 23.1 mmol), 4-boronic acid-phenol (3.51 g, 25.5 mmol), palladium-tetrakis(triphenylphosphine) (2.67 g, 2.31 mmol) and sodium hydrogen carbonate in DME were heated under microwave irradiation for 30 min. The reaction mixture was concentrated in vacuo and the remainder suspended between DCM and H₂O. The organic layer was separated, washed with H₂O and brine, dried (MgSO₄), filtered and concentrated in vacuo. Purification by column chromatography (DCM-MeOH, 1:0 to 3:2), followed by trituration with Et₂O afforded the title compound: RT=1.82 min; m/z (ES⁺)=230 [M+H]⁺(Method B).

### Preparation 30: 5-(4-Hydroxyphenyl)-3-methylpyridine-2-carboxylic acid ethylamide

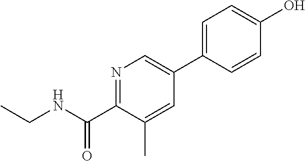

Ethylamine hydrochloride (158 mg, 1.94 mmol) was added to a solution of 5-(4-hydroxyphenyl)-3-methylpyridine-2-carboxylic acid (Preparation 29, 370 mg, 1.61 mmol), EDCI (372 mg, 1.94 mmol), HOBt (263 mg, 1.94 mmol) and DIPEA (620 μL, 3.54 mmol) in THF (10 mL) and the resulting reaction mixture was stirred at ambient temperature for 4 h. The solvent was removed in vacuo and the remainder was dissolved in EtOAc (150 mL), washed with H₂O (100 mL), saturated aqueous NaHCO₃ solution (100 mL) and brine, dried (MgSO₄), filtered and concentrated in vacuo to afford the title compound: RT=2.85 min; m/z (ES⁺)=257.07 [M+H]⁺.

### Preparation 31: 2-Bromo-5-[1-(4-isopropylbenzyl)azetidin-3-yloxy]pyridine

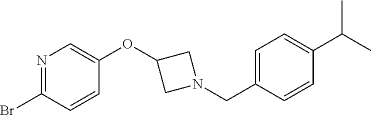

Methanesulfonylchloride (2.44 g, 31.6 mmol) was added to a solution of 1-(4-isopropylbenzyl)azetidin-3-ol (Preparation 2, 5.90 g, 28.7 mmol) and triethylamine (4.40 mL, 31.6 mmol) in THF (10 mL) at 0° C. The resulting reaction mixture was stirred at ambient temperature for 20 min before adding to a solution of 6-bromo-pyridin-3-ol (5.00 g, 28.7 mmol) and NaH (60% dispersion in oil, 4.06 g, 115 mmol) in DMF (15 mL). The resulting reaction mixture was heated at 60° C. for 16 h. The reaction mixture was diluted with EtOAc, washed with H₂O and brine, dried (MgSO₄), filtered and concentrated in vacuo. Purification by column chromotography (EtOAc-DCM, 1:4 to 3:7) afforded the title compound: RT=2.76 min; m/z (ES⁺)=361.27, 363.27 [M±H]⁺.

### Preparation 32: 4-{5-[1-(4-Isopropylbenzyl)azetidin-3-yloxy]pyridin-2-yl}benzoic acid methyl ester

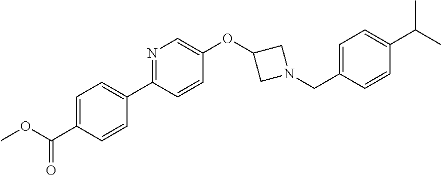

The title compound was synthesized from 2-bromo-5-[1-(4-isopropylbenzyl)azetidin-3-yloxy]pyridine (Preparation 31, 350 mg, 972 μmol) and 4-boronic acid-benzoic acid methyl ester (190 mg, 1.07 mmol) employing a procedure similar to that outlined in Example 9: RT=2.89 min; m/z (ES⁺)=417.27 [M±H]⁺.

### Preparation 33: 2-Fluoro-4-{5-[1-(4-isopropylbenzyl)azetidin-3-yloxy]pyridin-2-yl}benzoic acid ethyl ester

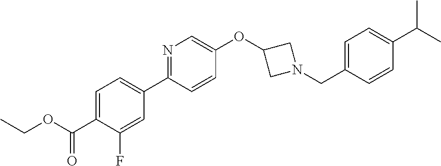

The title compound was synthesized from 2-bromo-5-[1-(4-isopropylbenzyl)azetidin-3-yloxy]pyridine (Preparation 31, 350 mg, 972 μmol) and 4-boronic acid-2-fluorobenzoic acid ethyl ester (210 mg, 1.069 mmol) employing a procedure similar to that outlined in Example 9: RT=3.13 min; m/z (ES⁺)=449.28 [M±H]⁺.

### Preparation 34: 4-Bromo-2-methylbenzoic acid methyl ester

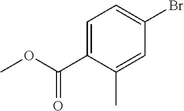

4-Bromo-2-methylbenzoic acid (32.56 mmol) was dissolved in 1:1 DCM/methanol (140 mL) and TMS-diazomethane (2M solution in diethyl ether, 65.12 mmol) was added dropwise until a yellow colour persisted. The mixture was quenched with acetic acid until the solution was de-colourised. The solution was concentrated under vacuum and the residue dissolved in DCM. This was washed with saturated NaHCO₃ (aq), and the aqueous extracted with further DCM. The organic extracts were combined, dried over MgSO₄ and concentrated to afford the title compound: δ_{H} (400 MHz, CDCl₃) 2.59 (s, 3H) 3.89 (s, 3H) 7.39 (d, J=8.20 Hz, 1H) 7.43 (s, 1H) 7.79 (d, J=8.20 Hz, 1H).

### Preparation 35: 2-Methyl-4-(4,4,5,5-tetramethyl-[1,3,2]dioxaborolan-2-yl)benzoic acid methyl ester

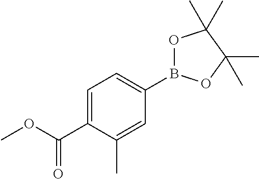

4-Bromo-2-methylbenzoic acid methyl ester (0.028 mmol), 4,4,5,5,4′,4′,5′,5′-octamethyl-[2,2′]bi[[1,3,2]dioxaborolanyl] (0.028 mmol) and potassium acetate (0.064 mmol) were combined in dioxane (65 mL) and de-gassed with argon. [1,1′-bis(diphenylphosphino)-ferrocene]dichloropalladium (5.62×10⁻⁴ mmol) and 1,1′-bis(diphenylphosphino)ferrocene (5.62×10⁻⁴ mmol) were added and the mixture stirred at 95° C. under argon for 16 h. The mixture was cooled then partitioned between water and ethyl acetate. The water was separated and washed with ethyl acetate. The organic extracts were combined, dried over MgSO₄, concentrated and purified by dry-flash chromatography to afford the title compound: RT=4.07 min; m/z (ES⁺)=277.24 [M+H]⁺.

### Preparation 36: 3-[6-(4-Methoxycarbonyl-3-methylphenyl)pyridin-3-yloxy]azetidine-1-carboxylic acid tert-butyl ester

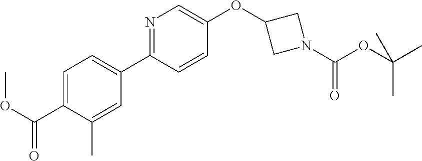

3-(6-Bromopyridin-3-yloxy)azetidine-1-carboxylic acid tert-butyl ester (17.79 mmol), 2-Methyl-4-(4,4,5,5-tetramethyl-[1,3,2]dioxaborolan-2-yl)-benzoic acid methyl ester (19.57 mmol) and cesium carbonate (26.69 mmol) were combined in 4:1 dioxane:H₂O (65 mL) and degassed with argon. Bis(diphenylphosphino)ferrocene]dichloropalladium (1.78 mmol) was added and the mixture stirred at 90° C. under argon for 16 h. The reaction mixture was cooled and partitioned between ethyl acetate and sat. Na₂CO₃ (aq). The aqueous layer was separated and extracted with further ethyl acetate. The organics were combined, dried over MgSO₄ and concentrated. The crude mixture was purified by flash column chromatography followed by trituration to afford the title compound: RT=4.07 min; m/z (ES⁺)=399.33 [M+H]⁺.

### Preparation 37: 4-[5-(Azetidin-3-yloxy)pyridin-2-yl]-2-methylbenzoic acid methyl ester, trifluoroacetic acid salt

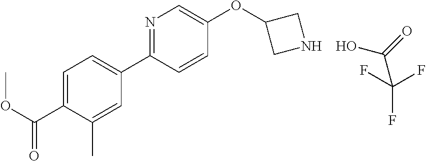

3-[6-(4-Methoxycarbonyl-3-methylphenyl)pyridin-3-yloxy]azetidine-1-carboxylic acid tert-butyl ester (9.80 mmol) was dissolved in DCM (32 mL), and TFA (8 mL) was added. The mixture was stirred at room temperature for 3 h before concentrating to dryness under vacuum to afford the title compound: RT=2.29 min; m/z (ES⁺)=299.22 [M+H]⁺.

### Preparation 38: 2-Methyl-4-{5-[1-(4-trifluoromethylbenzyl)azetidin-3-yloxy]pyridine-2-yl}benzoic acid methyl ester

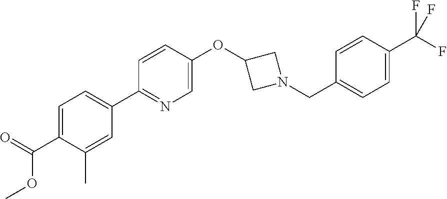

To a solution of 4-[5-(azetidin-3-yloxy)pyridin-2-yl]-2-methylbenzoic acid methyl ester, trifluoroacetic acid salt (4.9 mmol) in DCM (20 mL) was added triethylamine (9.8 mmol). After 5 min sodium triacetoxyborohydride (6.3 mmol) was added, and stirring continued for 17 h. The reaction mixture was diluted with DCM and washed with water and brine. The organic extract was dried (MgSO₄) and concentrated. The resulting solid was triturated with 1:1 diethyl ether/(?) to afford the title compound: RT=2.90 min; m/z (ES⁺)=457.31 [M+H]⁺.

### Preparation 39: 2-Methyl-4-{5-[1-(4-trifluoromethylbenzyl)azetidin-3-yloxy]pyridine-2-yl}benzoic acid, sodium salt

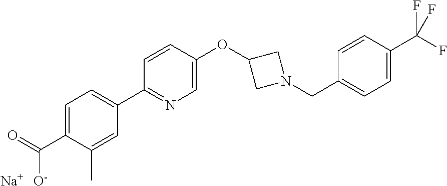

To a stirred suspension of 2-methyl-4-{5-[1-(4-trifluoromethylbenzyl)azetidin-3-yloxy]pyridine-2-yl}benzoic acid methyl ester (3.5 mmol) in methanol (20 mL) was added sodium hydroxide (4.2 mmol) in water (20 mL). The mixture was heated to 50° C. for 65 h. The reaction mixture was cooled and concentrated. The resulting solid was filtered and washed with water to obtain the title compound: RT=2.72, m/z (ES⁺) 443.30 [M+H]⁺.

### Preparation 40: 3-[6-(4-Methoxycarbonyl-3-fluorophenyl)pyridin-3-yloxy]azetidine-1-carboxylic acid tert-butyl ester

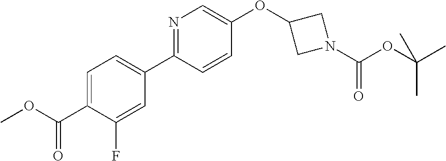

To a degassed solution of 2-bromo-5-[1-(3-isopropyl-[1,2,4]oxadiazol-5-ylmethyl)azetidin-3-yloxy]pyridine (23.5 mmol), 4-boronic acid-2-fluorobenzoic acid methyl ester (25.8 mmol) and cesium carbonate (70.4 mmol) in dioxane/water (4:1) 75 mL was added Pd(Cl)₂(dppf).DCM (2.3 mmol). The stirred mixture was heated to 95° C. for 16 h. The reaction mixture was cooled, concentrated and re-dissolved in ethyl acetate. The solution was filtered through celite and washed with saturated aqueous sodium carbonate (×2) and brine. The solution was dried (MgSO₄) and concentrated. Flash chromatography afforded the title compound: RT=3.93 min; m/z (ES⁺)=404.29 [M+H]⁺.

### Preparation 41: 3-[6-(4-Carboxy-3-fluorophenyl)pyridin-3-yloxy]azetidine-1-carboxylic acid tert-butyl ester

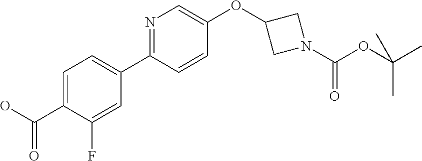

To a stirred solution of lithium hydroxide hydrate (73.4 mmol) in water (60 mL) was added 3-[6-(4-methoxycarbonyl-3-fluorophenyl)pyridin-3-yloxy]azetidine-1-carboxylic acid tert-butyl ester (14.7 mmol) in methanol (133 mL). The mixture was heated to 50° C. for 3 h, then allowed to cool and stand at room temperature for 64 h. The mixture was concentrated to remove the methanol, then diluted with further water (60 mL). The solution was extracted with diethyl ether, and the aqueous extract separated and acidified to pH 2 by careful addition of 6M hydrochloric acid. The resulting precipitate was filtered and dried. The filtrate was basified to pH 4 by addition of sodium hydroxide solution and extracted three times with ethyl acetate. The combined organic extracts were washed with brine, dried (MgSO₄) and concentrated to obtain a solid. Both solids were combined and comprised the title compound: RT 3.54=min; m/z (ES⁺)=390.27 [M+H]⁺.

### Preparation 42: 3-[6-(4-Carbamoyl-3-fluorophenyl)pyridin-3-yloxy]azetidine-1-carboxylic acid tert-butyl este

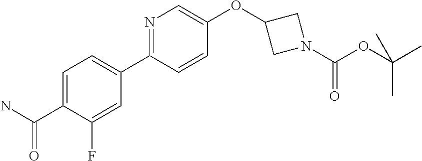

EDCI (18.4 mmol) and HOBt (18.4 mmol) were stirred in DMF (35 mL) until the reaction mixture became homogenous. 3-[6-(4-Carboxy-3-fluorophenyl)pyridin-3-yloxy]azetidine-1-carboxylic acid tert-butyl ester (14.7 mmol) was added and stirring continued for a further 2 h. Ammonia in dioxane (0.5M, 29.4 mmol) was added and the mixture stirred at room temperature for 16 h. Sodium hydroxide in methanol (7M, 5 mL) was added and after 30 min the mixture diluted with water and extracted with ethyl acetate. The combined organic extracts were washed with brine, dried and concentrated to obtain the title compound: RT 3.05=min; m/z (ES⁺)=388.29 [M+H]⁺.

### Preparation 43: 4-[5-(Azetidin-3-yloxy)pyridin-2-yl]-2-fluorobenzamide trifluoroacetate

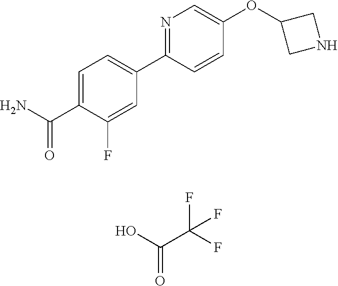

3-[6-(4-Carbamoyl-3-fluorophenyl)pyridin-3-yloxy]azetidine-1-carboxylic acid tert-butyl ester (5.67 mmol) was dissolved in DCM (40 mL) and TFA (10 mL) added. After stiffing for 16 h, the mixture was concentrated. The resulting residue was re-dissolved in DCM and concentrated. The residue was then dissolved in toluene and concentrated to afford the title compound: RT=1.90 min; m/z (ES⁺)=288.17 [M+H]⁺.

### Preparation 44: 2-Fluoro-4-{5-[1-(4-trifluoromethylbenzyl)azetidin-3-yloxy]pyridin-2-yl}-benzoic acid, sodium salt

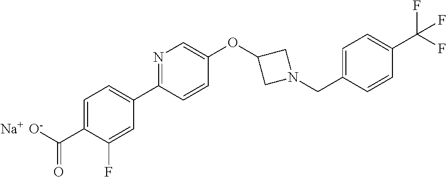

The title compound was synthesized from 2-fluoro-4-{5-[1-(4-trifluoromethylbenzyl)-azetidin-3-yloxy]pyridin-2-yl}benzoic acid ethyl ester (Preparation 27, 0.53 mmol) and sodium hydroxide (0.56 mmol) employing a procedure similar to that outlined in Preparation 16: RT=2.54 min; m/z (ES⁺)=447.24 [M+H]⁺.

### Example 1

### 4′-[1-(4-Isopropylbenzyl)azetidin-3-yloxy]biphenyl-4-carboxylic acid (2-hydroxy-1-hydroxymethylethyl)amide

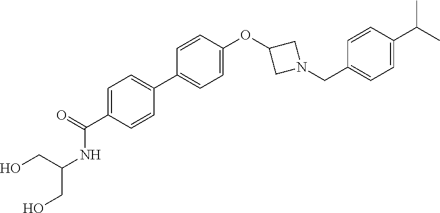

A flask was charged with 4′-[1-(4-isopropylbenzyl)azetidin-3-yloxy]biphenyl-4-carboxylic acid (0.26 mmol), WSC (0.29 mmol), 1-hydroxybenzotriazole (0.29 mmol) and DMF (5 mL) added, followed by triethylamine (0.26 mmol). After stirring for 30 min, the 2-aminopropane-1,3-diol (0.3 mmol) was added and stirring continued for 16 h. After this time a further WSC (0.29 mmol) was added, and the mixture heated to 50° C. for 8 h. The mixture was cooled, and purified by SCX followed by HPLC; RT=2.83 min; m/z (ES⁺)=475.27 [M+H]⁺

The examples listed in Table 1 were prepared employing a procedure similar to that outlined in Example 1:

TABLE 1

| Ex | Structure | Name | RT | m/z |
|---|---|---|---|---|
| 2 | 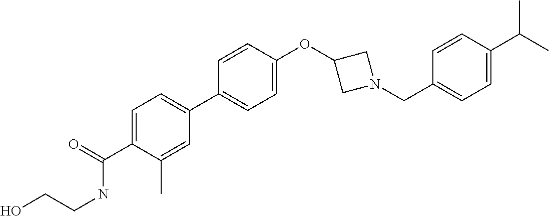 | 4′-[1-(4-Isopropyl- benzyl)azetidin-3-yloxy]-3- methylbiphenyl-4-carboxylic acid (2-hydroxyethyl)amide | 2.74 | 459.31 |
| 3 | 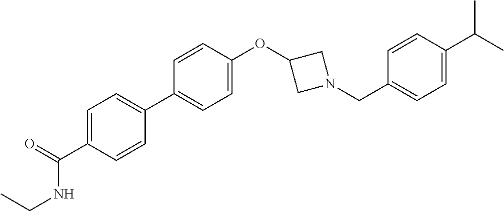 | 4′-[1-(4-Isopropyl- benzyl)azetidin-3- yloxy]biphenyl-4-carboxylic acid ethylamide | 3.06 | 429.33 |
| 4 | 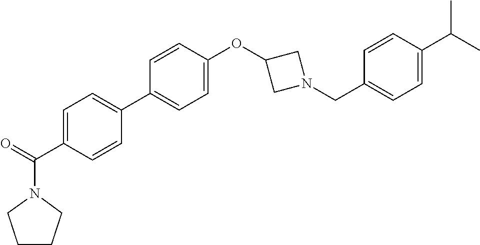 | {4′-[1-(4-Isopropyl- benzyl)azetidin-3- yloxy]biphenyl-4-yl}- pyrrolidin-1-yl-methanone | 3.13 | 455.27 |
| 5 | 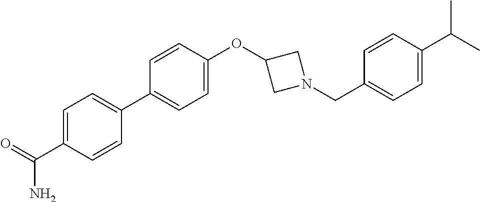 | 4′-[1-(4-Isopropyl- benzyl)azetidin-3- yloxy]biphenyl-4-carboxylic acid amide | 2.90 | 401.24 |
| 6 | 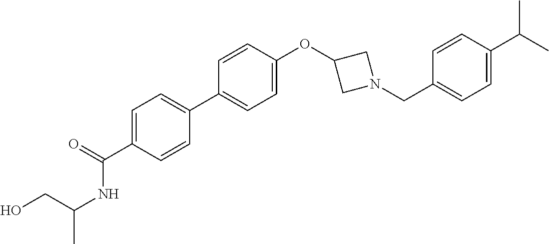 | 4′-[1-(4-Isopropyl- benzyl)azetidin-3- yloxy]biphenyl-4-carboxylic acid (2-hydroxy-l-methyl- ethyl)amide | 2.92 | 459.27 |
| 7 | 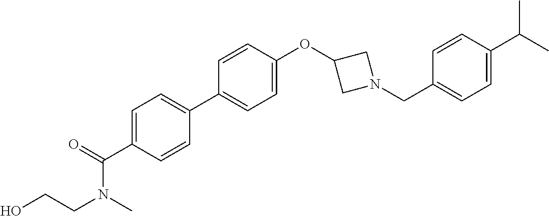 | 4′-[1-(4-Isopropyl- benzyl)azetidin-3- yloxy]biphenyl-4-carboxylic acid (2-hydroxy-l-methyl- ethyl)methylamide | 2.74 | 459.24 |
| 8 | 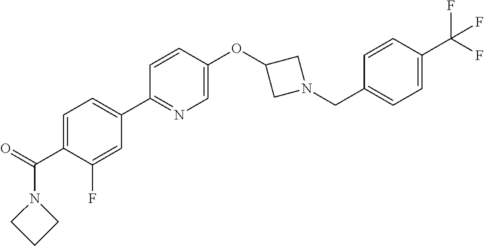 | Azetidine-1-yl-(2-fluoro-4-{5- [1-(4-trifluoromethylbenzyl)- azetidin-3-yloxy]pyridin-2- yl}phenyl)methanone | 2.63 | 486.29 |
| 9 | 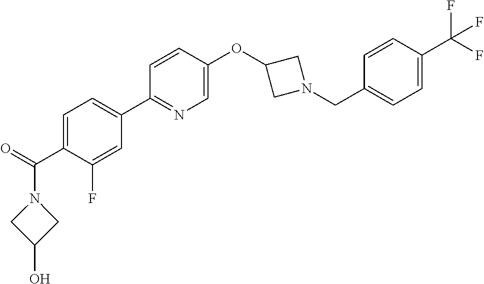 | (2-Fluoro-4-{5-[1-(4- trifluoromethylbenzyl)azetidin- 3-yloxy]pyridin-2-yl}phenyl)- (3-hydroxyazetidin-1-yl)- methanone | 2.52 | 502.29 |

### Example 10

### 4′-[1-(4-Trifluoromethylbenzyl)azetidin-3-yloxy]biphenyl-4-carboxylic acid ethylamide

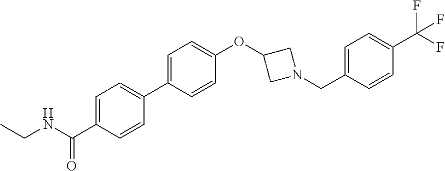

Dioxane:H₂O (4:1, 4 mL) was added to 3-(4-bromophenoxy)-1-(4-trifluoromethyl-benzyl)-azetidine (Preparation 11, 159 mg, 401 μmol), 4-boronic acid N-ethyl benzamide (87.0 mg, 455 μmol), cesium carbonate (200 mg, 620 μmol) and [1,1′-bis(diphenylphosphino)-ferrocene]dichloropalladium (34.0 mg, 41.0 μmol) and the resulting reaction mixture was degassed with argon and heated at 90° C. for 16 h. The solvent was removed in vacuo and the remainder was dissolved in DCM, washed with 1M NaOH and brine, dried (MgSO₄), filtered and concentrated in vacuo. Purification by column chromatography (EtOAc—IH, 7:3 to 1:0) afforded the title compound: RT=2.92 min; m/z (ES⁺)=455.13 [M+H]⁺.

The examples listed in Table 2 were prepared from 3-(4-bromophenoxy)-1-(4-trifluoromethylbenzyl)azetidine (Preparation 11) and the appropriate boronic acid employing a procedure similar to that outlined in Example 10:

TABLE 2

| Ex | Structure | Name | RT | m/z |
|---|---|---|---|---|
| 11 | 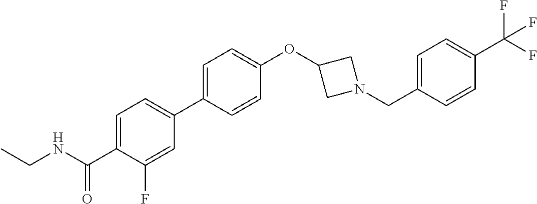 | 3-Fluoro-4'-[1-(4- trifluoromethylbenzyl)- azetidin-3-yloxy]biphenyl- 4-carboxylic acid ethylamide | 2.90 | 473.11 |
| 12 | 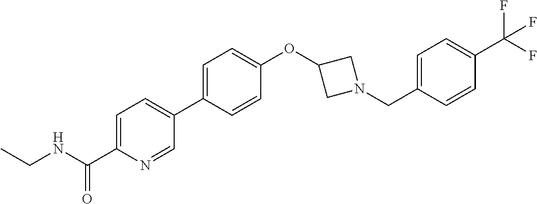 | 5-{4-[1-(4-Trifluoromethyl- benzyl)azetidin-3-yloxy]- phenyl}pyridine-2- carboxylic acid ethylamide | 2.88 | 456.11 |

### Example 13

### N-Ethyl-6-{4-[1-(4-trifluoromethylbenzyl)azetidin-3-yloxy]phenyl}nicotinamide

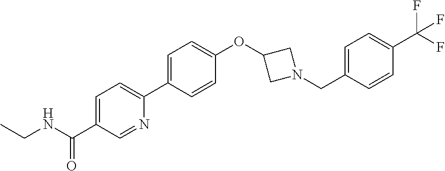

The title compound was synthesized from 3-[4-(4,4,5,5-tetramethyl-[1,3,2]dioxaborolan-2-yl)phenoxy]-1-(4-trifluoromethylbenzyl) azetidine (Preparation 13, 80.0 mg, 180 μmol) and 6-bromo-N-ethyl-nicotinamide (42.0 mg, 180 μmol) employing a procedure similar to that outlined in Example 10: RT=2.70 min; m/z (ES⁺)=456.41 [M+H]⁺.

### Example 14

### (2-Methyl-6-{4-[1-(4-trifluoromethylbenzyl)azetidin-3-yloxy]phenyl}pyridin-3-yl)pyrrolidin-1-yl-methanone

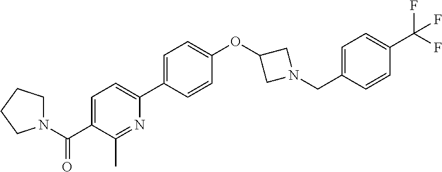

A solution of 2-methyl-6-{4-[1-(4-trifluoromethylbenzyl)azetidin-3-yloxy]phenyl}-nicotinic sodium carboxylate (Preparation 16, 100 mg, 220 μmol), EDCI (62.0 mg, 720 μmol), HOBt (44.0 mg, 320 μmol) and triethylamine (60.0 μL, 430 μmol) was stirred at 50° C. for 20 min, prior to the addition of pyrrolidine (27 μL, 320 μmol). The resulting reaction mixture was stirred at 50° C. for 16 h. The solvent was removed in vacuo and the remainder was dissolved in EtOAc (50 mL), washed with H₂O (3×10 mL) and saturated aqueous Na₂CO₃ solution (3×10 mL), dried (MgSO₄), filtered and concentrated in vacuo. Purification by column chromatography (EtOAc—NEt₃, 99:1), followed by trituration in Et₂O afforded the title compound: RT=2.59 min; m/z (ES⁺)=496.36 [M+H]⁺.

### Example 15

### N-Ethyl-2-fluoro-4-{5-[1-(4-trifluoromethylbenzyl)azetidin-3-yloxy]pyridin-2-yl}benzamide

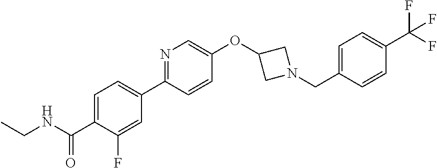

The title compound was synthesized from 2-bromo-5-[1-(4-trifluoromethylbenzyl)-azetidin-3-yloxy]pyridine (Preparation 18, 150 mg, 390 μmol) and 4-boronic acid-N-ethyl-2-fluoro-benzamide (90.0 mg, 430 μmol) employing a procedure similar to that outlined in Example 10: RT=2.63 min; m/z (ES⁺)=464.06 [M+H]⁺.

### Example 16

### (2-Methyl-4-{5-[1-(4-trifluoromethylbenzyl)azetidin-3-yloxy]pyridin-2-yl}-phenyl)pyrrolidin-1-ylmethanone

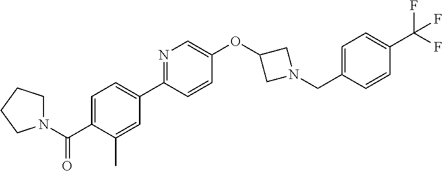

The title compound was synthesized from 2-bromo-5-[1-(4-trifluoromethylbenzyl)-azetidin-3-yloxy]pyridine (Preparation 18, 110 mg, 280 μmol) and [2-methyl-4-(4,4,5,5-tetramethyl-[1,3,2]dioxaborolan-2-yl)phenyl]pyrrolidin-1-ylmethanone (Preparation 19, 99.0 mg, 310 μmol) employing a procedure similar to that outlined in Example 10: RT=2.65 min; m/z (ES⁺)=496.39 [M+H]⁺.

### Example 17

### N-Ethyl-2-methyl-4-{5-[1-(4-trifluoromethylbenzyl)azetidin-3-yloxy]pyridin-2-yl}benzamide

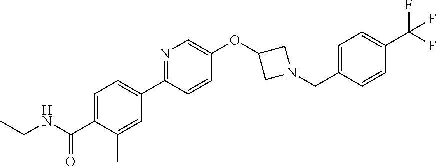

The title compound was synthesized from 2-bromo-5-[1-(4-trifluoromethylbenzyl)-azetidin-3-yloxy]pyridine (Preparation 18, 100 mg, 260 μmol) and N-ethyl-2-methyl-4-(4,4,5,5-tetramethyl-[1,3,2]dioxaborolan-2-yl)benzamide (Preparation 21, 82.0 mg, 280 μmol) employing a procedure similar to that outlined in Example 10: RT=2.68 min; m/z (ES⁺)=470.33 [M+H]⁺.

### Example 18

### 4-(5-{1-[4-(1,1-Difluoroethyl)benzyl]azetidin-3-yloxy-pyridin-2-yl)-2-fluoro-N-(2-hydroxyethyl)benzamide

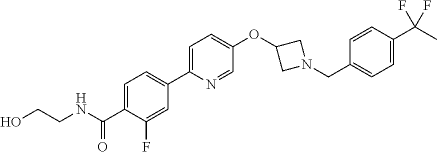

Sodium triacetoxyborohydride (146 mg, 690 μmol) was added to a solution of 4-[5-(azetidin-3-yloxy)pyridin-2-yl]-2-fluoro-N-(2-hydroxyethyl)benzamide hydrochloride (Preparation 25, 175 mg, 530 μmol) and 4-(1,1-difluoroethyl)benzaldehyde (90.0 mg, 530 μmol) in DCE (10 mL) and the resulting reaction mixture was stirred at ambient temperature for 16 h. The reaction mixture was diluted with DCM, washed with saturated aqueous NaHCO₃ solution (×2), dried (MgSO₄), filtered and concentrated in vacuo. Trituration in Et₂O afforded the title compound: RT=2.42 min; m/z (ES⁺)=486.33 [M+H]⁺.

### Example 19

### N-Ethyl-2-fluoro-4-{6-[1-(4-trifluoromethylbenzyl)azetidin-3-yloxy]pyridin-3-yl}benzamide

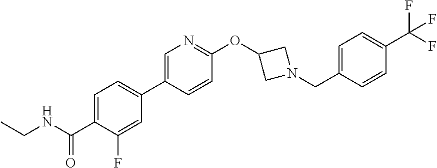

The title compound was synthesized from 5-bromo-2-[1-(4-trifluoromethylbenzyl)-azetidin-3-yloxy]pyridine (Preparation 26, 200 mg, 517 μmol) and N-ethyl-2-fluoro-4-(4,4,5,5-tetramethyl-[1,3,2]dioxaborolan-2-yl)benzamide (160 mg, 568 μmol) employing a procedure similar to that outlined in Example 10: RT=2.77 min; m/z (ES⁺)=474.15 [M+H]⁺.

### Example 20

### 2-Fluoro-N-(2-hydroxyethyl)-4-{5-[1-(4-trifluoromethylbenzyl)azetidin-3-yloxy]-pyridin-2-yl}benzamide

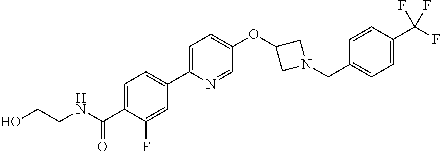

2-Fluoro-4-{5-[1-(4-trifluoromethylbenzyl)azetidin-3-yloxy]pyridin-2-yl}benzoic acid ethyl ester (Preparation 27, 115 mg, 24.2 mmol) in ethanolamine (5 mL) was heated at 100° C. for 16 h. The reaction mixture was cooled to ambient temperature and partitioned between EtOAc (150 mL) and H₂O (75 mL). The organic phase was separated, washed with saturated aqueous NaHCO₃ solution (50 mL) and brine, dried (MgSO₄), filtered and concentrated in vacuo. Purification by column chromatography (EtOAc-MeOH, 19:1) afforded the title compound: RT=2.52 min; m/z (ES⁺)=490.38 [M+H]⁺.

### Example 21

### (2-Fluoro-4-{5-[1-(4-trifluoromethylbenzyl)azetidin-3-yloxy]pyridin-2-yl}-phenyl)pyrrolidin-1-ylmethanone

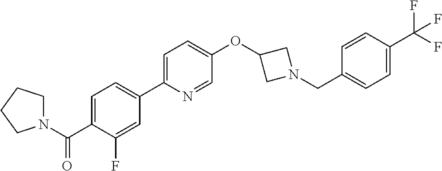

Trimethyl aluminium (2M, 270 μL, 530 μmol) was added dropwise to a solution of pyrrolidine (44.0 μL, 530 μmol) in toluene (2 mL) at 0° C. The reaction mixture was allowed to warm to ambient temperature prior to the addition of 2-fluoro-4-{5-[1-(4-trifluoromethyl-benzyl)azetidin-3-yloxy]pyridin-2-yl}benzoic acid ethyl ester (Preparation 27, 125 mg, 260 μmol) in toluene (2 mL). The resulting reaction mixture was heated at 110° C. for 16 h, cooled to ambient temperature and quenched with aqueous ammonium chloride solution. Purification by column chromatography (DCM-MeOH, 49:1) afforded the title compound: RT=2.86 min; m/z (ES⁺)=499.50 [M+H]⁺.

### Example 22

### 2-Fluoro-4-{5-[1-(4-trifluoromethylbenzyl)azetidin-3-yloxy]pyridin-2-yl}-benzamide

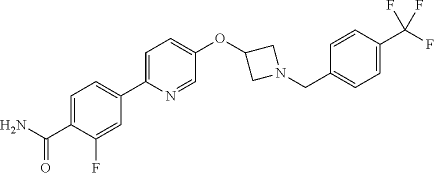

2-Fluoro-4-{5-[1-(4-trifluoromethylbenzyl)azetidin-3-yloxy]pyridin-2-yl}benzoic acid ethyl ester (Preparation 27, 100 mg, 21.1 mmol), 2M ammonia in MeOH (5 mL) and concentrated ammonia (1 mL) were heated in a sealed tube at 100° C. for 16 h. The solvent was removed in vacuo and the remainder was dissolved in EtOAc (100 mL), washed with H₂O (50 mL), saturated aqueous NaHCO₃ solution (50 mL) and brine, dried (MgSO₄), filtered and concentrated in vacuo. Purification by recrystallisation (1H-MeOH), followed by column chromatography (DCM-MeOH, 97:3) afforded the title compound: RT=2.64 min; m/z (ES⁺)=446.34 [M+H]⁺.

### Example 23

### N-Ethyl-2-fluoro-N-methyl-4-{5-[1-(4-trifluoromethylbenzyl)azetidin-3-yloxy]-pyridin-2-yl}benzamide

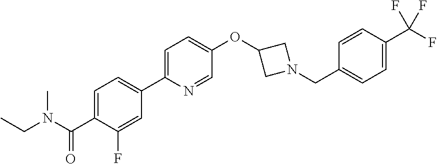

The title compound was synthesized from 2-fluoro-4-{-[1-(4-trifluoromethylbenzyl)-azetidin-3-yloxy]pyridin-2-yl}benzoic acid ethyl ester (Preparation 27, 120 mg, 250 μmol) and ethyl-methyl-amine (43.0 μL, 510 μmol) employing a procedure similar to that outlined in Example 21: RT=2.74 min; m/z (ES⁺)=488.35 [M+H]⁺.

### Example 24

### N-(2-Hydroxyethyl)-4-{6-[1-(4-trifluoromethylbenzyl)azetidin-3-yloxy]pyridin-3-yl}benzamide

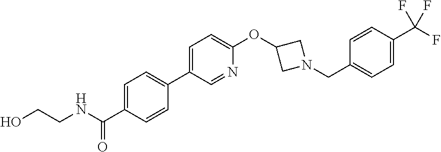

Potassium tertiary butoxide (155 mg, 1.38 mmol) was added to a solution of 4-(6-fluoropyridin-3-yl)benzoic acid (Preparation 28, 150 mg, 690 μmol) and 1-(4-trifluoromethyl-benzyl)azetidin-3-ol (Preparation 10, 160 mg, 690 μmol) in DMSO (2.5 mL) and the resulting reaction mixture was stirred at ambient temperature for 16 h. Ethanolamine (50.0 μL, 830 μmol), DIPEA (260 μL, 1.52 mmol) and HATU (316 mg, 830 μmol) were added to the reaction mixture and the resulting solution was stirred at ambient temperature for 16 h. The reaction mixture was diluted with DCM (200 mL), washed with H₂O (100 mL), saturated aqueous Na₂CO₃ solution and brine, dried (MgSO₄), filtered and concentrated in vacuo. Purification by column chromotography (EtOAc-MeOH, 47:3) afforded the title compound: RT=2.71 min; m/z (ES⁺)=472.33 [M+H]⁺.

### Example 25

### 3-Methyl-5-{4-[1-(4-trifluoromethylbenzyl)azetidin-3-yloxy]phenyl}pyridine-2-carboxylic acid ethylamide

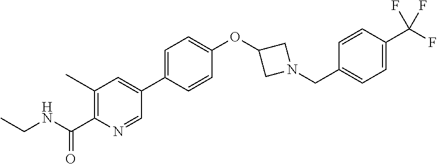

Potassium tertiary butoxide (45.0 mg, 40.0 μmol) was added to a solution of 5-(4-hydroxyphenyl)-3-methylpyridine-2-carboxylic acid ethylamide (Preparation 30, 100 mg, 390 μmol) in DMSO (5 mL) and the resulting reaction mixture was stirred at rt for 20 min. Methanesulfonic acid 1-(4-trifluoromethylbenzyl)azetidin-3-yl ester (Preparation 17, 125 mg, 430 μmol) in DMSO (2 mL) was added dropwise and the resulting reaction mixture was stirred at 100° C. for 16 h. The reaction mixture was diluted with EtOAc (50 mL), washed with H₂O and brine, dried (MgSO₄), filtered and concentrated in vacuo. Purification by column chromatography (EtOAc—IH, 7:3) afforded the title compound: RT=2.93 min; m/z (ES⁺)=470.37 [M+H]⁺.

### Example 26

### N-(2-Hydroxyethyl)-4-{5-[1-(4-isopropylbenzyl)azetidin-3-yloxy]pyridin-2-yl}-benzamide

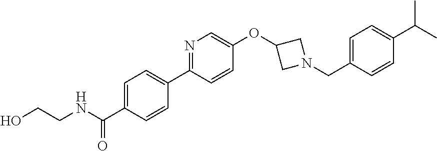

4-{5-[1-(4-Isopropylbenzyl)azetidin-3-yloxy]pyridin-2-yl}benzoic acid methyl ester (Preparation 32, 100 mg, 240 μmol) and ethanolamine (2 mL) in MeOH (3 mL) were heated in a sealed tube at 100° C. for 16 h. The reaction mixture was cooled to rt and the resulting crystals were collected by filtration and washed with H₂O to afford the title compound: RT=2.43 min; m/z (ES⁺)=446.34 [M+H]⁺.

### Example 27

### 2-Fluoro-N-(2-hydroxyethyl)-4-{5-[1-(4-isopropylbenzyl)azetidin-3-yloxy]-pyridin-2-yl}benzamide

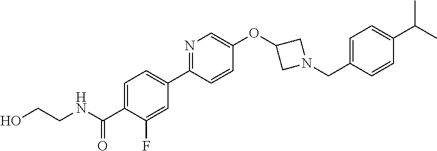

The title compound was synthesized from 2-fluoro-4-{5-[1-(4-isopropylbenzyl)-azetidin-3-yloxy]pyridin-2-yl}benzoic acid ethyl ester (Preparation 33, 100 mg, 223 μmol) employing a procedure similar to that outlined in Example 26: RT=2.59 min; m/z (ES⁺)=464.40 [M+H]⁺.

The examples listed in Table 3 were prepared from: 4-[5-(azetidin-3-yloxy)pyridin-2-yl]-2-fluorobenzamide trifluoroacetate (Preparation 43) and the appropriate aldehyde employing a procedure similar to that outlined in Example 18. In some cases, examples were purified by preparative HPLC.

TABLE 3

| Ex | Structure | Name | RT | m/z |
|---|---|---|---|---|
| 28 | 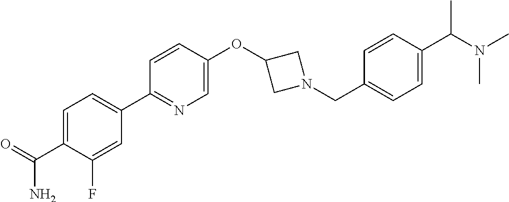 | 2-Methyl-4-{5-[1-(4- trifluoromethylbenzyl)- azetidin-3-yloxy]- pyridin-2-yl}benzamide | 1.84 | 449.34 |
| 29 | 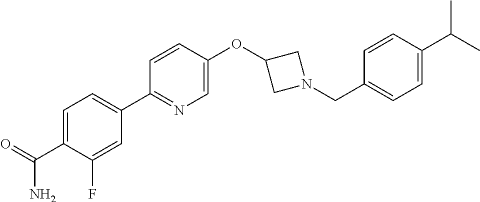 | 2-Fluoro-4-{5-[1-(4- isopropylbenzyl)- azetidin-3-yloxy]- pyridin-2-yl}benzamide | 2.67 | 420.34 |
| 30 | 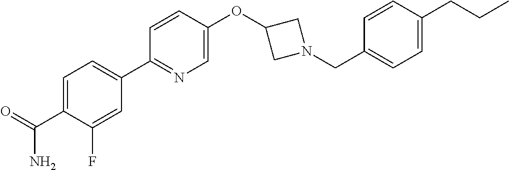 | 2-Fluoro-4-{5-[1-(4- propylbenzyl)azetidin-3- yloxy]pyridin-2-yl}- benzamide | 2.77 | 420.34 |
| 31 | 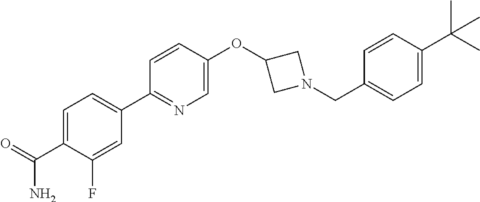 | 4-{5-[1-(4-tert- Butylbenzyl-)azetidin-3- yloxy]pyridin-2-yl}-2- fluorobenzamide | 2.79 | 434.36 |
| 32 | 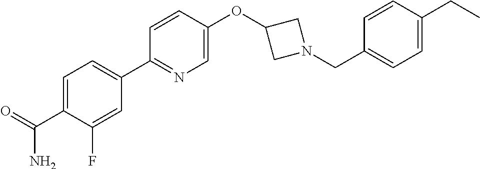 | 4-{5-[1-(4- Ethylbenzyl)azetidin-3- yloxy]pyridin-2-yl}-2- fluorobenzamide | 2.57 | 406.34 |
| 33 | 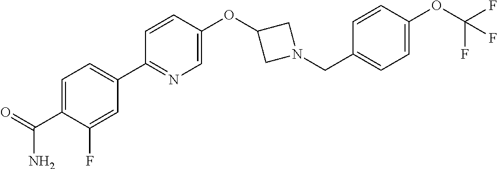 | 2-Fluoro-4-{5-[1-(4- trifluoromethoxy- benzyl)azetidin-3-yloxy]- pyridin-2-yl}benzamide | 2.67 | 462.30 |

The examples listed in Table 4 were prepared from 2-methyl-4-{5-[1-(4-trifluoromethylbenzyl)azetidin-3-yloxy]pyridine-2-yl}benzoic acid, sodium salt (Preparation 39) and the appropriate amine employing a procedure similar to that outlined in Example 14. In all cases, examples were purified by preparative HPLC.

TABLE 4

| Ex | Structure | Name | RT | m/z |
|---|---|---|---|---|
| 34 | 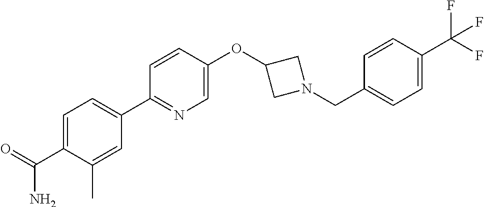 | 2-Methyl-4-{5-[1-(4- trifluoromethylbenzyl)- azetidin-3-yloxy]- pyridin-2-yl}benzamide | 2.50 | 442.32 |
| 35 | 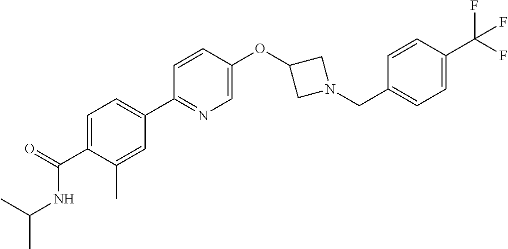 | N-Isopropyl-2-Methyl-4- {5-[1-(4-trifluoromethyl- benzyl)azetidin-3-yloxy]- pyridin-2-yl}benzamide | 2.75 | 484.39 |
| 36 | 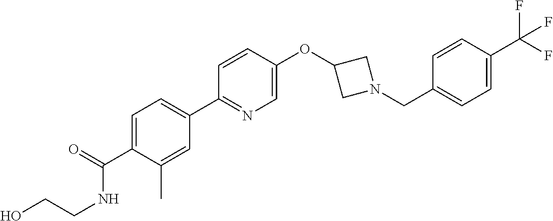 | N-(2-Hydroxyethyl-2- methyl-4-{5-[1-(4- trifluoromethyl- benzyl)azetidin-3-yloxy]- pyridin-2-yl}benzamide | 2.48 | 486.31 |
| 37 | 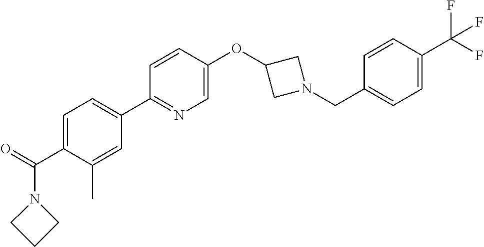 | Azetidin-1-yl-(2-methyl)- 4-{5-[1-(4-trifluoro- methylbenzyl)azetidin-3- yloxy]pyridin-2-yl}- methanone | 2.77 | 482.31 |
| 38 | 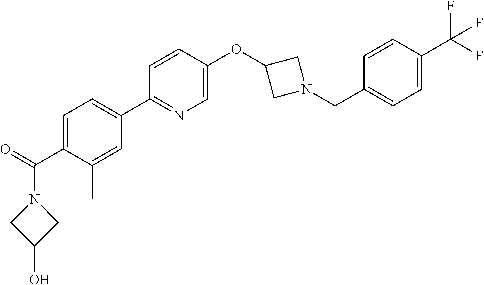 | (3-Hydroxyazetidin-1-yl- (2-methyl)-4-{5-[1-(4- trifluoromethyl- benzyl)azetidin-3-yloxy]- pyridin-2-yl}methanone | 2.54 | 498.29 |
| 39 | 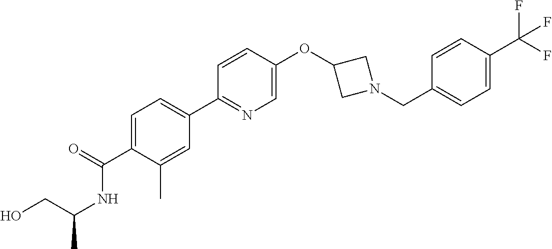 | N-((S)-2-Hydroxy-1- methylethyl)-(2-methyl)- 4-{5-[1-(4-trifluoro- methylbenzyl)azetidin- 3-yloxy]pyridin-2-yl}- benzamide | 2.59 | 500.38 |
| 40 | 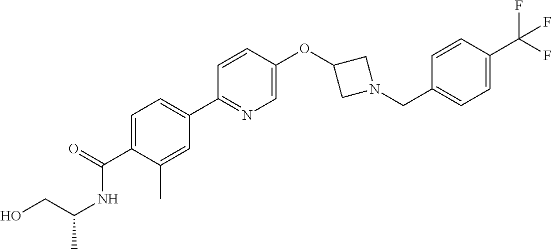 | N-((R)-2-Hydroxy-1- methylethyl)-(2-methyl)- 4-{5-[1-(4-trifluoro- methylbenzyl)azetidin-3- yloxy]pyridin-2-yl}- benzamide | 2.67 | 500.37 |
| 41 | 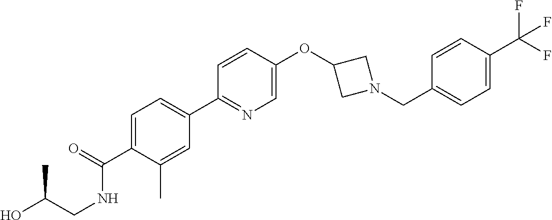 | N-((S)-2-Hydroxy- propyl)-(2-methyl)-4-{5- [1-(4-trifluoromethyl- benzyl)azetidin-3-yloxy]- pyridin-2-yl}benzamide | 2.55 | 500.38 |
| 42 | 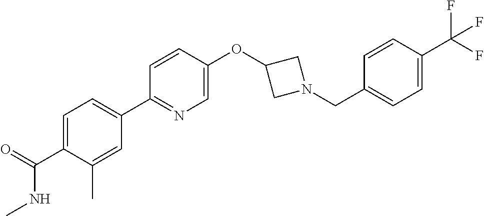 | 2,N-Dimethyl-4-{5-[1-(4- trifluoromethylbenzyl)- azetidin-3-yloxy]pyridin- 2-yl}benzamide | 2.63 | 456.33 |

The biological activity of the compounds of the invention may be tested in the following assay systems:

### Yeast Reporter Assay

The yeast cell-based reporter assays have previously been described in the literature (e.g. see Miret J. J. et al, 2002, J. Biol. Chem., 277:6881-6887; Campbell R. M. et al, 1999, Bioorg. Med. Chem. Lett., 9:2413-2418; King K. et al, 1990, Science, 250:121-123); WO 99/14344; WO 00/12704; and U.S. Pat. No. 6,100,042). Briefly, yeast cells have been engineered such that the endogenous yeast G-alpha (GPA1) has been deleted and replaced with G-protein chimeras constructed using multiple techniques. Additionally, the endogenous yeast GPCR, Step 3 has been deleted to allow for heterologous expression of a mammalian GPCR of choice. In the yeast, elements of the pheromone signaling transduction pathway, which are conserved in eukaryotic cells (for example, the mitogen-activated protein kinase pathway), drive the expression of Fus1. By placing β-galactosidase (LacZ) under the control of the Fus1 promoter (Fus1p), a system has been developed whereby receptor activation leads to an enzymatic read-out.

Yeast cells were transformed by an adaptation of the lithium acetate method described by Agatep et al, (Agatep, R. et al, 1998, Transformation of Saccharomyces cerevisiae by the lithium acetate/single-stranded carrier DNA/polyethylene glycol (LiAc/ss-DNA/PEG) protocol. Technical Tips Online, Trends Journals, Elsevier). Briefly, yeast cells were grown overnight on yeast tryptone plates (YT). Carrier single-stranded DNA (10 m), 2 μg of each of two Fus1p-LacZ reporter plasmids (one with URA selection marker and one with TRP), 2 μg of GPR119 (human or mouse receptor) in yeast expression vector (2 μg origin of replication) and a lithium acetate/polyethylene glycol/TE buffer was pipetted into an Eppendorf tube. The yeast expression plasmid containing the receptor/no receptor control has a LEU marker. Yeast cells were inoculated into this mixture and the reaction proceeds at 30° C. for 60 min. The yeast cells were then heat-shocked at 42° C. for 15 min. The cells were then washed and spread on selection plates. The selection plates are synthetic defined yeast media minus LEU, URA and TRP (SD-LUT). After incubating at 30° C. for 2-3 days, colonies that grow on the selection plates were then tested in the LacZ assay.

In order to perform fluorimetric enzyme assays for β-galactosidase, yeast cells carrying the human or mouse GPR119 receptor were grown overnight in liquid SD-LUT medium to an unsaturated concentration (i.e. the cells were still dividing and had not yet reached stationary phase). They were diluted in fresh medium to an optimal assay concentration and 90 μl of yeast cells added to 96-well black polystyrene plates (Costar). Compounds, dissolved in DMSO and diluted in a 10% DMSO solution to 10× concentration, were added to the plates and the plates placed at 30° C. for 4 h. After 4 h, the substrate for the β-galactosidase was added to each well. In these experiments, Fluorescein di (β-D-galactopyranoside) was used (FDG), a substrate for the enzyme that releases fluorescein, allowing a fluorimetric read-out. 20 μl per well of 500 μM FDG/2.5% Triton X100 was added (the detergent was necessary to render the cells permeable). After incubation of the cells with the substrate for 60 min, 20 μl per well of 1M sodium carbonate was added to terminate the reaction and enhance the fluorescent signal. The plates were then read in a fluorimeter at 485/535 nm.

The compounds of the invention give an increase in fluorescent signal of at least ˜1.5-fold that of the background signal (i.e. the signal obtained in the presence of 1% DMSO without compound). Compounds of the invention which give an increase of at least 5-fold may be preferred.

cAMP Assay

A stable cell line expressing recombinant human GPR119 was established and this cell line may be used to investigate the effect of compounds of the invention on intracellular levels of cyclic AMP (cAMP). The cell monolayers are washed with phosphate buffered saline and stimulated at 37° C. for 30 min with various concentrations of compound in stimulation buffer plus 1% DMSO. Cells are then lysed and cAMP content determined using the Perkin Elmer AlphaScreen™ (Amplified Luminescent Proximity Homogeneous Assay) cAMP kit. Buffers and assay conditions are as described in the manufacturer's protocol.

### In Vivo Feeding Study

The effect of compounds of the invention on body weight and food and water intake may be examined in freely-feeding male Sprague-Dawley rats maintained on reverse-phase lighting. In such a test, compounds of the invention and reference compounds are dosed by appropriate routes of administration (e.g. intraperitoneally or orally) and measurements made over the following 24 h. Rats are individually housed in polypropylene cages with metal grid floors at a temperature of 21±4° C. and 55±20% humidity. Polypropylene trays with cage pads are placed beneath each cage to detect any food spillage Animals are maintained on a reverse phase light-dark cycle (lights off for 8 h from 09.30-17.30 h) during which time the room was illuminated by red light. Animals have free access to a standard powdered rat diet and tap water during a two week acclimatization period. The diet is contained in glass feeding jars with aluminum lids. Each lid has a 3-4 cm hole in it to allow access to the food. Animals, feeding jars and water bottles are weighed (to the nearest 0.1 g) at the onset of the dark period. The feeding jars and water bottles are subsequently measured 1, 2, 4, 6 and 24 h after animals are dosed with a compound of the invention and any significant differences between the treatment groups at baseline compared to vehicle-treated controls.

Compounds of the invention showing a hypophagic effect at one or more time points at a dose of ≦100 mg/kg may be preferred.

Anti-Diabetic Effects of Compounds of the Invention in an In-Vitro Model of Pancreatic Beta cells (HIT-T15)

### Cell Culture

HIT-T15 cells (passage 60) can be obtained from ATCC, and cultured in RPMI1640 medium supplemented with 10% fetal calf serum and 30 nM sodium selenite. All experiments should be done with cells at less than passage 70, in accordance with the literature, which describes altered properties of this cell line at passage numbers above 81 (Zhang H J, Walseth T F, Robertson R P. Insulin secretion and cAMP metabolism in HIT cells. Reciprocal and serial passage-dependent relationships. Diabetes. 1989 January; 38(1):44-8).

cAMP Assay

HIT-T15 cells were plated in standard culture medium in 96-well plates at 100,000 cells/0.1 ml/well and cultured for 24 hr and the medium was then discarded. Cells were incubated for 15 min at room temperature with 100 μl stimulation buffer (Hanks buffered salt solution, 5 mM HEPES, 0.5 mM IBMX, 0.1% BSA, pH 7.4). This was discarded and replaced with compound dilutions over the range 0.001, 0.003, 0.01, 0.03, 0.1, 0.3, 1, 3, 10, 30 μM in stimulation buffer in the presence of 0.5% DMSO. Cells were incubated at room temperature for 30 min. Then 75 ul lysis buffer (5 mM HEPES, 0.3% Tween-20, 0.1% BSA, pH 7.4) was added per well and the plate was shaken at 900 rpm for 20 min. Particulate matter was removed by centrifugation at 3000 rpm for 5 min, then the samples were transferred in duplicate to 384-well plates, and processed following the Perkin Elmer AlphaScreen cAMP assay kit instructions. Briefly 25 μl reactions were set up containing 8 μl sample, 5 μl acceptor bead mix and 12 μl detection mix, such that the concentration of the final reaction components is the same as stated in the kit instructions. Reactions were incubated at room temperature for 150 min, and the plate was read using a Packard Fusion instrument. Measurements for cAMP were compared to a standard curve of known cAMP amounts (0.01, 0.03, 0.1, 0.3, 1, 3, 10, 30, 100, 300, 1000 nM) to convert the readings to absolute cAMP amounts. Data was analysed using XLfit 3 software.

Representative compounds of the invention were found to increase cAMP at an EC₅₀ of less than 10 μM. Compounds showing an EC₅₀ of less than 1 μM in the cAMP assay may be preferred.

### Insulin Secretion Assay

HIT-T15 cells are plated in standard culture medium in 12-well plates at 106 cells/1 mL/well and cultured for 3 days and the medium then discarded. Cells are washed ×2 with supplemented Krebs-Ringer buffer (KRB) containing 119 mM NaCl, 4.74 mM KCl, 2.54 mM CaCl₂, 1.19 mM MgSO₄, 1.19 mM KH2PO₄, 25 mM NaHCO₃, 10 mM HEPES at pH 7.4 and 0.1% bovine serum albumin. Cells are incubated with 1 mL KRB at 37° C. for 30 min which is then discarded. This is followed by a second incubation with KRB for 30 min, which is collected and used to measure basal insulin secretion levels for each well. Compound dilutions (0, 0.1, 0.3, 1, 3, 10 uM) are then added to duplicate wells in 1 mL KRB, supplemented with 5.6 mM glucose. After 30 min incubation at 37° C. samples are removed for determination of insulin levels. Measurement of insulin may be done using the Mercodia Rat insulin ELISA kit, following the manufacturers instructions, with a standard curve of known insulin concentrations. For each well insulin levels are subtracted by the basal secretion level from the pre-incubation in the absence of glucose. Data may be analysed using XLfit 3 software.

Compounds showing an EC₅₀ of less than 1 μM in the insulin secretion assay may be preferred.

### Oral Glucose Tolerance Tests

The effects of compounds of the invention on oral glucose (Glc) tolerance were evaluated in male Sprague-Dawley rats. Food was withdrawn 16 h before administration of Glc and remained withdrawn throughout the study. Rats had free access to water during the study. A cut was made to the animals' tails, then blood (1 drop) was removed for measurement of basal Glc levels 60 min before administration of the Glc load. Then, the rats were weighed and dosed orally with test compound or vehicle (20% aqueous hydroxypropyl-β-cyclodextrin) 45 min before the removal of an additional blood sample and treatment with the Glc load (2 g kg⁻¹ p.o.). Blood samples were then taken from the cut tip of the tail 5, 15, 30, 60, 120, and 180 min after Glc administration. Blood glucose levels were measured just after collection using a commercially available glucose-meter (OneTouch® Ultra™ from Lifescan). Representative compounds of the invention statistically reduced the Glc excursion at doses of ≦10 mg kg⁻¹.

The effects of compounds of the invention on oral glucose (Glc) tolerance may also be evaluated, for example in male C57B1/6 or male ob/ob mice. Food may be withdrawn 5 h before administration of Glc and remain withdrawn throughout the study. Mice should have free access to water during the study. A cut may be made to the animals' tails, then blood (20 μL) may be removed for measurement of basal Glc levels 45 min before administration of the Glc load. Subsequently, the mice are weighed and dosed orally with test compound or vehicle (20% aqueous hydroxypropyl-β-cyclodextrin or 25% aqueous Gelucire 44/14) 30 min before the removal of an additional blood sample (20 μL) and treatment with the Glc load (2-5 g kg⁻¹ p.o.). Blood samples (20 μL) may then be taken 25, 50, 80, 120, and 180 min after Glc administration. The 20 μL blood samples for measurement of Glc levels are taken from the cut tip of the tail into disposable micro-pipettes (Dade Diagnostics Inc., Puerto Rico) and the sample should be added to 480 μL of haemolysis reagent. Duplicate 20 μL aliquots of the diluted haemolysed blood are then added to 180 μL of Trinders glucose reagent (Sigma enzymatic (Trinder) colorimetric method) in a 96-well assay plate. After mixing, the samples are left at room temperature for 30 min before being read against Glc standards (Sigma glucose/urea nitrogen combined standard set). Compounds of the invention of particular interest will typically result in a statistically significant reduction of the Glc excursion at doses ≦100 mg kg⁻¹ in this test.

1. A compound of formula (I): 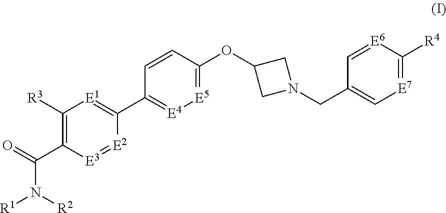
wherein:
E¹, E² and E³ are CH except that up to one of E¹, E² and E³ can be N;
E⁴ and E⁵ are CH except that up to one of E⁴ and E⁵ can be N;
E⁶ and E⁷ are independently CH or N;
R¹ and R² are independently selected from hydrogen, C₁₋₄ alkyl optionally substituted by one or more hydroxy groups, and a 4- to 6-membered heterocyclic ring containing one heteroatom selected from N and O; or R¹ and R² together with the N to which they are attached may form a 4- to 6-membered heterocyclic ring optionally containing a further heteroatom selected from N and O and optionally substituted by one or more hydroxy or C₁₋₄ alkyl groups;
R³ is hydrogen, halo or methyl; and
R⁴ is C₁₋₄ alkyl or C₁₋₄ alkoxy, either of which may be substituted by one or more fluoro groups;
or a pharmaceutically acceptable salt thereof.

2. A compound according to claim 1, or a pharmaceutically acceptable salt thereof, wherein E¹, E² and E³ are CH.

3. A compound according to claim 2, or a pharmaceutically acceptable salt thereof, wherein E⁴ is CH.

4. A compound according to claim 2, or a pharmaceutically acceptable salt thereof, wherein E⁴ is N.

5. A compound according to claim 4, or a pharmaceutically acceptable salt thereof, wherein E⁵ is CH.

6. A compound according to claim 5, or a pharmaceutically acceptable salt thereof, wherein E⁶ and E⁷ are CH.

7. A compound according to claim 6, or a pharmaceutically acceptable salt thereof, wherein R¹ and R² are independently selected from hydrogen and C₁₋₄ alkyl optionally substituted by one or more hydroxy groups; or R¹ and R² together with the N to which they are attached may form a 5- or 6-membered heterocyclic ring optionally containing a further heteroatom selected from N and O and optionally substituted by one or more hydroxy or C₁₋₄ alkyl groups.

8. A compound according to claim 7, or a pharmaceutically acceptable salt thereof, wherein R³ is hydrogen or methyl.

9. A compound according to claim 8, or a pharmaceutically acceptable salt thereof, wherein R⁴ is C₁₋₄ alkyl.

10. A compound according to claim 8, or a pharmaceutically acceptable salt thereof, wherein R⁴ is isopropyl.

11. A compound according to claim 8, or a pharmaceutically acceptable salt thereof, wherein R⁴ is C₁₋₂ alkyl optionally substituted with one or more fluorine atoms.

12. A compound according to claim 8, or a pharmaceutically acceptable salt thereof, wherein R⁴ is trifluoromethyl.

13. A compound according to claim 1, selected from any one of Examples 1 to 42, as the free base or a pharmaceutically acceptable salt thereof.

14. A pharmaceutical composition comprising a compound according to claim 1, or a pharmaceutically acceptable salt thereof and a pharmaceutically acceptable carrier.

15. (canceled)

16. A method for the treatment of a disease or condition in which GPR119 plays a role comprising administering to a subject in need thereof an effective amount of a compound according to claim 1, or a pharmaceutically acceptable salt thereof.

17. A method for the regulation of satiety comprising administering to a subject in need thereof an effective amount of a compound according to claim 1, or a pharmaceutically acceptable salt thereof.

18. A method for the treatment of obesity comprising administering to a subject in need thereof an effective amount of a compound according to claim 1, or a pharmaceutically acceptable salt thereof.

19. A method for the treatment of diabetes comprising administering to a subject in need thereof an effective amount of a compound according to claim 1, or a pharmaceutically acceptable salt thereof.

20. A method for the treatment of metabolic syndrome (syndrome X), impaired glucose tolerance, hyperlipidemia, hypertriglyceridemia, hypercholesterolemia, low HDL levels or hypertension comprising administering to a patient in need thereof an effective amount of a compound according to claim 1, or a pharmaceutically acceptable salt thereof.
